(19) United States
(12) Patent Application Publication
(10) Pub. No.: US 2013/0316958 A1
(43) Pub. Date: Nov. 28, 2013

(54) HIGHLY POTENT PEPTIDES TO CONTROL CANCER AND NEURODEGENERATIVE DISEASES

(71) Applicant: Jae U. Jung, Los Angeles, CA (US)

(72) Inventor: Jae U. Jung, Los Angeles, CA (US)

(73) Assignee: University of Southern California

(21) Appl. No.: 13/812,482

(22) PCT Filed: Jul. 26, 2011
(86) PCT No.: PCT/US11/45406

§ 371 Date: Aug. 1, 2013

Related U.S. Application Data
(60) Provisional application No. 61/368,466, filed on Jul. 28, 2010.

(51) Int. Cl. C07K 14/47; C07K 16/08; C07K 14/005; C07K 16/18; G01N 33/50

(52) U.S. Cl. CPC C07K 14/4747; C07K 16/18; G01N 33/5011; C07K 14/005; C07K 16/081
USPC 514/18.9; 435/375; 530/327; 530/328; 530/387.9; 435/7.1

(57) ABSTRACT

This invention provides compositions and method of diminishing or inhibiting autophagy by administering a FLIP protein that binds to Atg3, interfering with the formation of the LC3-Atg4-Atg7-Atg3 conjugation complex necessary for autophagy induction. This invention also provides FLIP peptide fragments that promote or induce autophagy by interfering with the activity of FLIP.

### CROSS-REFERENCE TO RELATED APPLICATIONS

This application claims the benefit under 35 U.S.C. §119(e) of U.S. Provisional Application No. 61/368,466, filed on Jul. 28, 2010, the contents of which are hereby incorporated by reference in their entirety into the present disclosure.

### FIELD OF THE INVENTION

The present invention relates to the use of FLIP proteins to regulate autophagy by inhibiting the conjugation of LC3 ubiquitin-like protein to Atg3 E2-like enzyme. The present invention further relates to the composition, isolation and use of FLIP-derived peptide fragments to induce growth suppression and autophagic death.

### BACKGROUND OF THE INVENTION

Autophagy is an active homeostatic degradation process of removal or turnover of cytoplasmic components from a cell. In autophagy, a double-layered membrane called a phagophore expands and engulfs cytoplasm and organelles and forms an autophagosome.

The autophagosome then fuses with a lysosome, which provides hydrolytic enzymes that break down the cargo of the autophagosome, resulting in the degradation and recycling of the cargo. Although autophagy occurs at basal levels in most tissues to effect routine cellular housekeeping, it can also be induced at higher levels as a cell-survival mechanism in response to different forms of metabolic stress, such as nutrient depletion, absence of growth factors, or low oxygen levels. (Deretic and Klionsky (2008) Sci. Am. 298:74-81; Levine and Kroemer (2008) Cell 132:27-42; Shintani and Klionsky (2004) Science 306:990-995).

While autophagy has a cytoprotective role, paradoxically autophagy may also contribute to cell damage and cell death. For example, autophagy is believed to be involved in both the promotion and prevention of cancer. In precancerous cells, autophagy may act as a suppressor of cancer. (Shintani and Klionsky (2004) Science 306:990-995). Likewise, inhibition of autophagy in precancerous cells may allow those cells to survive and grow. (Gozuacik and Kimchi (2004) Oncogene 23:2891-2906). However, at later stages autophagy may promote tumor cell survival and growth. As a tumor grows, cancer cells may utilize autophagy in order to survive nutrient-limited and low-oxygen conditions, particularly in poorly vascularized regions of a tumor, as autophagy can be induced by conditions such as nutrient depletion or low oxygen levels. (Shintani and Klionsky (2004) Science 306:990-995). Additionally, autophagy may protect cancer cells against certain types of therapeutic treatments. For example, radiation or chemotherapy treatments that are aimed at killing cancer cells may instead induce higher levels of autophagy, resulting in the removal of damaged organelles such as mitochondria before they can trigger apoptosis (programmed cell death). (Deretic and Klionsky (2008) Sci. Am. 298:74-81).

Consequently, regulation of autophagy is a promising strategy for the prevention and treatment of cancers. In precancerous stages, treatments enhancing autophagy in cells at risk of cancer could decrease the likelihood of accumulating mutations and developing secondary tumors by removing DNA-damaging molecules before they can accumulate in the cells. Conversely, at later stages, treatments inhibiting autophagy in cancerous cells could inhibit tumor cell growth by preventing the cells from breaking down cell components in order to survive low oxygen or nutrient-limiting conditions. Treatments inhibiting autophagy could also be beneficial for preventing cancerous cells undergoing certain types of radiation or chemotherapy treatment from removing cellular components that would trigger apoptosis. (Deretic and Klionsky (2008) Sci. Am. 298:74-81).

Misregulation of autophagy is also implicated in neurodegenerative diseases such as Alzheimer's disease, Parkinson's disease, Huntington's disease, and transmissible spongiform encephalopathies. Data showing the accumulation of autophagosomes in the brains of patients with these disorders led to the initial hypothesis that autophagy contributed to the pathogenesis of these diseases. However, more recent studies have suggested that autophagy may actually protect against neurodegenerative diseases, and that the accumulation of autophagosomes in patients having these diseases represents either the activation of autophagy as a beneficial physiological response or the consequence of a defect in autophagosomal maturation. (Levine and Kroemer (2008) Cell 132:27-42). For example, it is believed that an inability of autophagosomes to complete their development into mature form may result in a buildup of amyloid beta protein fragments in the projections of neurons, which may contribute to Alzheimer's disease. (Deretic and Klionsky (2008) Sci. Am. 298:74-81). Autophagy is also implicated in the clearance of aggregate-prone mutant proteins associated with various other neurodegenerative diseases. Pharmacological activation of autophagy reduces levels of soluble and aggregated forms of mutant Huntington protein, proteins mutated in spinocerebellar ataxia, mutant forms of α-synuclein, and mutant tau and also reduces these proteins' cellular toxicity in vitro and their neurotoxicity in either mouse or Drosophila models. (Rubinsztein et al. (2007) Nat. Rev. Drug Discov. 6:304-312).

Consequently, regulation of autophagy is a promising strategy for the treatment of neurogenerative diseases. Treatments enhancing autophagy in diseased nerve cells could prevent neuronal cell death by removing toxic aggregates of proteins accumulated in those cells. (Deretic and Klionsky (2008) Sci. Am. 298:74-81).

Autophagy is also implicated in the process of pathogen infection. For example, autophagy-like structures appear after viral infection, and in the case of herpes simplex virus, autophagy is induced following infection through the activation of a double-stranded RNA-activated protein kinase R. However, during viral replication, autophagy is not induced, because the protein kinase R that induces autophagy is inactivated. (Shintani and Klionsky (2004) Science 306:990-995). It is also suggested that microbial virulence may be determined in part by the ability of pathogens to successfully antagonize host autophagy. (Levine and Kroemer (2008) Cell 132:27-42). Consequently, induction of autophagy is a promising strategy for eliminating replicating intracellular viruses.

Genetic analysis in yeast has identified more than 20 autophagy-related genes (Atg) that encode proteins necessary for the induction of autophagy and for the generation, maturation, and recycling of autophagosomes. (Levine and Kroemer (2008) Cell 132:27-42). During the process of autophagosome maturation, the LC3 ubiquitin-like protein, a mammalian homolog of yeast Atg8, is cleaved by the Atg4 cysteine protease to expose its C-terminal Gly. The cleaved LC3 protein is then activated by the Atg7 E1-like enzyme and is transferred to the Atg3 E2-like enzyme. Finally, LC3 is covalently conjugated to phosphatidylethanolamine and embedded into lipid membranes for autophagic vesicle expansion. (Levine and Klionsky (2004) Dev. Cell 6:463-477). While the specific role of the LC3-Atg4-Atg3-Atg7 conjugation reaction is not known, it is essential for autophagosome formation.

FLICE-like inhibitor proteins (FLIP proteins) are proteins that contain two death effector domains, DED1 and DED2. Several FLIPs have been identified, including cellular FLIP (cFLIP) and viral FLIP of Kaposi's sarcoma-associated herpes virus (KSHV-vFLIP), Herpes virus saimiri (HVS-vFLIP), and Molluscum contagiosum virus (MCV). FLIPs have been shown to protect cells against death receptor-mediated apoptosis by interacting with Fas-associated death-domain-containing protein (FADD) through their DEDs in order to disrupt the death-inducing signaling complex. (Thome and Tschopp (2001) Nat. Rev. Immunol. 1:50-58). KSHV-vFLIP has also been shown to interact with the IKKαβγ complex and the Tumor Necrosis Factor (TNF) Receptor Associated Factor proteins (TRAFs) and also constitutively activates the NF-κB pathway, which contributes to its anti-apoptotic functions. (Matta and Chaudhary (2004) Proc. Natl. Acad. Sci. USA 101:9399-9404; Guasparri et al. (2006) EMBO Rep. 7:114-119). However, prior to this work it was not known whether FLIPs were implicated in autophagy.

### SUMMARY OF THE INVENTION

Applicants have discovered that cellular FLIP (cFLIP) and viral FLIPs (vFLIPs or vFLIP) compete against LC3 for binding to Atg3 protein, and diminish or inhibit the formation of the LC3-Atg4-Atg7-Atg3 conjugation complex that is necessary for autophagy induction. Applicants also have identified isolated peptide fragments of vFLIP and cFLIP, variants, hybrids and biological equivalents of these fragment polypeptides that inhibit or diminish the ability of cFLIP and/or vFLIPs to bind to Atg3 and thereby promote formation of the LC3-Atg4-Atg7-Atg3 conjugation complex thereby leading to robust autophagy induction and autophagic cell death. Applicants have further identified the regions of the Atg3 protein that interact with the vFLIP and cFLIP peptide fragments.

Accordingly, this invention provides methods and compositions to augment or promote autophagy in a cell, tissue or subject in need thereof by administering an effective amount of one or more cFLIP and/or vFLIP peptide fragment, variant, hybrid or biological equivalent of each thereof to the cell, tissue or subject. Exemplary peptide fragments having this function or activity are provided in Tables 1, Table 3 and 6, along with their respective sequence listing identifier numbers (SEQ ID NOs). Diseases or pathological conditions that would benefit from promoting or augmenting autophagy are identified herein and known in the art. Equivalent polypeptides having a predetermined sequence homology or identity to the specific sequence identified below are further provided herein. Polynucleotides encoding these polypeptides are also provided herein and can be used in these methods in addition to or as an alternative to administration of the polypeptides. Sequences of exemplary polynucleotides are provided in Tables 5 and 7 along with their respective sequence listing identifier numbers and the identity of the peptide which the specific nucleotide sequence encodes.

In another aspect this invention provides anti-autophagy methods and compositions. The compositions compete with LC3 in the formation of the LC3-Atg4-Atg7-Atg3 conjugation complex that is necessary for autophagy induction. The methods require administering an effective amount of vFLIP and/or cFLIP protein or polypeptide that competes against LC3 for the binding of Atg3 and subsequent complex formation. Diseases or pathological conditions that would benefit from the administration of anti-autophagy compositions and methods are identified herein and known in the art and include for example the treatment of solid tumors and cancers beyond the pre-cancerous stage. In one aspect, they are co-administered with other anticancer agents such as chemotherapy. Exemplary polypeptides are identified in Table 2 along with their respective sequence listing identifiers. Accordingly the administration of an effective amount of cFLIP and/or vFLIPs inhibit autophagy in a cell, tissue or subject to which the polypeptides have been administered. Polynucleotides encoding these polypeptides are also provided herein and can be used in these methods in addition to or as an alternative to administration of the polypeptides.

TABLE 1

| SEQ ID NO. | AMINO ACID SEQUENCE (Native Source) |
|---|---|
| 1 | EVVLFLLNVF (α2 region of KSHV vFLIP: amino acids 20-29) |
| 2 | QTFLHWVYCMEN (α4 region of KSHV vFLIP: amino acids 128-139) |
| 3 | EMLLFLCRDV (α2 region of cFLIP Short: amino acids 19-28) |
| 4 | KSFLDLVVELEK (α4 region of cFLIP Short: amino acids 128-139) |
| 5 | YCLLFLINGC (α2 region of HVS vFLIP: amino acids 20-29) |
| 6 | SSVILCVFSNML (α4 region of HVS vFLIP: amino acids 128-139) |
| 7 | SLLLFLCHDA (α2 region of MCV vFLIP: amino acids 26-35) |
| 8 | SRFVELVLALEN (α4 region of MCV vFLIP: amino acids 134-145) |

TABLE 2

| SEQ | |
|---|---|
| ID NO. | AMINO ACID SEQUENCE (Native Source) |
| 9 | MSAEVIHQVEEALDTDEKEMLLFLCRDVAIDVVPPNVRDLLD |
| | ILRERGKLSVGDLAELLYRVRRFDLLKRILKMDRKAVETHLL |
| | RNPHLVSDYRVLMAEIGEDLDKSDVSSLIFLMKDYMGRGKIS |
| | KEKSFLDLVVELEKLNLVAPDQLDLLEKCLKNIHRIDLKTKI |
| | KQYKQSVQGAGTSYRNVLQAAIQKSLKDPSNNFRLHNGRSKE |
| | QRLKEQLGAQQEPVKKSIQESEAFLPQSIPEERYKMKSKPLG |
| | ICLIIDCIGNETELLRDTFTSLGYEVQKFLHLSMHGISQILG |
| | QFACMPEHRDYDSFVCVLVSRGGSQSVYGVDQTHSGLPLHHI |
| | RRMFMGDSCPYLAGKPKMFFIQNYVVSEGQLEDSSLLEVDGP |
| | AMKNVEFKAQKRGLCTVHREADFFWSLCTADMSLLEQSHSSP |
| | SLYLQCLSQKLRQERKRPLLDLHIELNGYMYDWNSRVSAKE |
| | KYYVWLQHTLRKKLILSYT |
| | (cFLIP Long “cFLIP_{L}”) |
| 10 | MATYEVLCEVARKLGTDDREVVLFLLNVFIPQPTLAQLIGAL |
| | RALKEEGRLTFPLLAECLFRAGRRDLLRDLLHLDPRFLERHL |
| | AGTMSYFSPYQLTVLHVDGELCARDIRSLIFLSKDTIGSRST |
| | PQTFLHWVYCMENLDLLGPTDVDALMSMLRSLSRVDLQRQVQ |
| | TLMGLHLSGPSHSQHYRHTP |
| | (KSHV vFLIP) |
| 11 | MDLKTTVLHITDSFTEEEMYCLLFLINGCIPRNCNAVKISDL |
| | IIETLSKSTQWDICLTQCLYVLRKIELLLNLFQVTKEDVKQS |
| | FFTQLQLETHVLTLVNVNNNLTAKDEKRLCFILDQFFPRNVV |
| | ASSVILCVFSNMLCEMPVLECLCQLKKCLKQIGRSDLAKTV |
| | (HVS vFLIP) |
| 12 | MSDSKEVPSLPFLRHLLEELDSHEDSLLLFLCHDAAPGCTTV |
| | TQALCSLSQQRKLTLAALVEMLYVLQRMDLLKSRFGLSKEGA |
| | EQLLGTSFLTRYRKLMVCVGEELDSSELRALRLFACNLNPSL |
| | STALSESSRFVELVLALENVGLVSPSSVSVLADMLRTLRRLD |
| | LCQQLVEYEQQEQARYRYCYAASPSLPVRTLRRGHGASEHEQ |
| | LCMPVQESSDSPELLRTPVQESSSDSPEQTT |
| | (MCV vFLIP) |
| 13 | MSAEVIHQVEEALDTDEKEMLLFLCRDVAIDVVPPNVRDLLD |
| | ILRERGKLSVGDLAELLYRVRRFDLLKRILKMDRKAVETHLL |
| | RNPHLVSDYRVLMAEIGEDLDKSDVSSLIFLMKDYMGRGKIS |
| | KEKSFLDLVVELEKLNLVAPDQLDLLEKCLKNIHRIDLKTKI |
| | QKYKQSVQGAGTSYRNVLQAAIQKSLKDPSNNFRMITPYAHC |
| | PDLKILGNCSM |
| | (cFLIP Short “cFLIPs”) |

TABLE 3

| | D-ISOMER RETRO-INVERSO PEPTIDES |
|---|---|
| SEQ ID NO. | (Native Source) |
| 14 | RRRQRRKKRGY-G (TAT-DOMAIN) |
| 15 | RRRQRRKKRGY-G-FVNLLFLVVE (TAT-α2) |
| 16 | RRRQRRKKRGY-G-FVNLAAAVVE (TAT-α2m) |
| 17 | RRRQRRKKRGY-G-NEMCYVWHLFTQ (TAT-α2) |
| 18 | RRRQRRKKRGY-G-NEMCAAAHAATQ (TAT-α4M) |

In still another aspect, this invention provides methods of increasing or inducing the death of a precancerous cell, a malignant cell such as a cancer cell and/or increasing or inducing cancer cell death, eliminating viral particles associated with a viral infection, and/or treating or ameliorating the symptoms of cancer or alternatively, neurodegenerative diseases such as Alzheimer's disease, Parkinson's disease, Huntington's disease, and neuronal degeneration, by administering an effective amount of an isolated vFLIP or cFLIP peptide fragment, variant, hybrid or biological equivalent of each thereof, or polynucleotide encoding any one or more of these, that compete against vFLIP or cFLIP for binding of Atg3, resulting in inducing or increasing autophagy. In one aspect, the peptide fragment, variant, hybrid or biological equivalent of each thereof is conjugated to a transduction domain such as HIV TAT. In other aspect, the peptide is the retro-inverso form alone or in combination with the cell penetrating sequence such as HIV TAT. In one aspect, an effective amount of a polynucleotide encoding the fragment is administered to a cell, a tissue or a subject in need thereof.

When peptides or peptide fragments are administered, they can be combined or contained within a suitable vector such as one containing a transduction domain. Accordingly, the invention provides the isolated polypeptides or isolated peptide fragments operatively linked to a suitable transduction domain such as the TAT domain (e.g., see Table 3). One or more retro-inverso versions of the peptides shown in Table 1 are identified in Table 3 or retro-inverso versions of variant and hybrid peptides identified in Table 6 and can be administered in the methods of this invention alone or in combination with a transduction domain to facilitate cell entry. In an alternative aspect, the peptides and peptide fragments further contain a detectable label. Compositions and host cells containing the peptide fragments are further provided herein. Suitable host cells include prokaryotic as well as eukaryotic cells. Examples of prokaryotic cells include bacterial cells such as E. coli. Suitable eukaryotic cells include, but are not limited to yeast cells, insect cells and mammalian cells such as human cells. The cells may be primary cells or cultured cell lines.

When polynucleotides encoding polypeptide or peptide fragments, variants, hybrids, or biological equivalents of each thereof, are administered, the polynucleotides can be combined or contained within a suitable vector such as an expression or replication vector. Sequences of exemplary polynucleotides are provided in Table 5 and Table 7 along with their respective sequence listing identifier numbers and the identity of the peptide which the specific nucleotide sequence encodes. Accordingly, the invention provides the isolated polynucleotides operatively linked to regulatory elements necessary for replication and/or expression in a suitable host cell or tissue. In an alternative embodiment, the polynucleotides are conjugated or linked to a detectable label.

Compositions and host cells containing the isolated polynucleotides as described above are further provided herein. Suitable host cells include prokaryotic as well as eukaryotic cells. Examples of prokaryotic cells include bacterial cells such as E. coli. Suitable eukaryotic cells include, but are not limited to yeast cells, insect cells and mammalian cells such as human cells. The cells may be primary cells or cultured cell lines. In yet a further aspect, the polynucleotides are isolated from the host cells. The isolated host cells when containing the isolated polynucleotide also are useful for expressing the polynucleotide which in another aspect, is isolated from the isolated host cell.

Also provided by this invention are isolated Atg3 peptide fragments that bind to α2 and α4 peptide cFLIP and/or vFLIP fragments. The peptides comprise amino acids 193-268 and/or 268-315 regions of Atg3 and are identified in Table 4. The Atg3 peptide fragments can be combined or contained within a suitable vector such as one containing a transduction domain. Accordingly, the invention provides the isolated polypeptides or isolated peptide fragments operatively linked to a suitable transduction domain or cell penetrating sequence such as the TAT domain (see Table 3). One or more retro-inverso versions of the Atg3 peptides are further provided by this invention. In an alternative aspect, the peptides and peptide fragments further contain a detectable label.

Compositions and host cells containing the peptide fragments are further provided herein. Suitable host cells include prokaryotic as well as eukaryotic cells. Examples of prokaryotic cells include bacterial cells such as E. coli. Suitable eukaryotic cells include, but are not limited to yeast cells, insect cells and mammalian cells such as human cells. The cells may be primary cells or cultured cell lines.

This invention also provides isolated polynucleotides encoding the proteins and peptide fragments, hybrids, variants and equivalents of each thereof, such as the Atg3 peptide fragments (see e.g., Table 4). Sequences of exemplary polynucleotides are provided in Table 5 and 7 along with their respective sequence listing identifier numbers and the identity of the peptide which the specific nucleotide sequence encodes. The isolated polynucleotides can be combined or contained within a suitable vector such as an expression or replication vector. Accordingly, the invention provides the isolated polynucleotides operatively linked to regulatory elements necessary for replication and/or expression in a suitable host cell or tissue. In an alternative embodiment, the polynucleotides are conjugated or linked to a detectable label.

Compositions and host cells containing the isolated polynucleotides and/or peptides as described above are further provided herein. Suitable host cells include prokaryotic as well as eukaryotic cells. Examples of prokaryotic cells include bacterial cells such as E. coli. Suitable eukaryotic cells include, but are not limited to yeast cells, insect cells and mammalian cells such as human cells. The cells may be primary cells or cultured cell lines. In yet a further aspect, the polynucleotides are isolated from the host cells.

TABLE 4

| SEQ ID NO. | AMINO ACID SEQUENCE (Native Source) |
|---|---|
| 19 | ILQTRTYDLYITYDKYYQTPRLWLFGYDEQRQPLTV |
| | EHMYEDISQDHVKKTVTIENHPHLPPPPMCSVHPCR |
| | HAEV (Atg3 Binding Domain) |
| 20 | VMKKIIETVAEGGGELGVHMYLLIFLKFVQAVIPTI |
| | EYDYTRHFTM (Atg3 Binding Domain) |

TABLE 5

| SEQ ID NO. | NUCLEIC ACID CONSENSUS SEQUENCE |
|---|---|
| 21 | gargtngtnytnttyytnytnaaygtntty (α2 region of KSHV vFLIP: amino |
| | acids 20-29) |
| 22 | caracnttyytncaytgggtntaytgyatggaraay (α4 region of KSHV vFLIP: |
| | amino acids 128-139) |
| 23 | garatgytnytnttyytntgymgngaygtn (α2 region of cFLIP Short: amino |
| | acids 19-28) |
| 24 | aarwsnttyytngayytngtngtngarytngaraar (α4 region of cFLIP Short: |
| | amino acids 128-139) |
| 25 | taytgyytnytnttyytnathaayggntgy (α2 region of HVS vFLIP: amino |
| | acids 20-29) |
| 26 | wsnwsngtnathytntgygtnttywsnaayatgytn (α4 region of HVS vFLIP: |
| | amino acids 128-139) |
| 27 | wsnytnytnytnttyytntgycaygaygcn (α2 region of MCV vFLIP: amino |
| | acids 26-35) |
| 28 | wsnmgnttygtngarytngtnytngcnytngaraay (α4 region of MCV vFLIP: |
| | amino acids 134-145) |
| 29 | atgwsngcngargtnathcaycargtngargargcnytngayacngaygaraargaratgytnyt |
| | nttyytntgymgngaygtngcnathgaygtngtnccnccnaaygtnmgngayytnytngayat |
| | hytnmgngarmgnggnaarytnwsngtnggngayytngcngarytnytntaymgngtnmg |
| | nmgnttygayytnytnaarmgnathytnaaratggaymgnaargcngtngaracncayytnyt |
| | nmgnaayccncayytngtnwsngaytaymgngtnytnatggcngarathggngargayytng |
| | ayaarwsngaygtnwsnwsnytnathttyytnatgaargaytayatgggnmgnggnaarathw |
| | snaargaraarwsnttyytngayytngtngtngarytngaraarytnaayytngtngcnccngayc |
| | arytngayytnytngaraartgyytnaaraayathcaymgnathgayytnaaracnaarathcara |
| | artayaarcarwsngtncarggngcnggnacnwsntaymgnaaygtnytncargcngcnathc |
| | araarwsnytnaargayccnwsnaayaayttymgnytncayaayggnmgnwsnaargarcar |
| | mgnytnaargarcarytnggngcncarcargarccngtnaaraarwsnathcargarwsngarg |
| | cnttyytnccncarwsnathccngargarmgntayaaratgaarwsnaarccnytnggnathtgy |
| | ytnathathgaytgyathggnaaygaracngarytnytnmgngayacnttyacnwsnytnggnt |
| | aygargtncaraarttyytncayytnwsnatgcayggnathwsncarathytnggncarttygcnt |
| | gyatgccngarcaymgngaytaygaywsnttygtntgygtnytngtnwsnmgnggnggnws |
| | ncarwsntntayggngtngaycaracncaywsnggnytnccnytncaycayathmgnmgn |
| | atgttyatgggngaywsntgyccntayytngcnggnaarccnaaratgttgttgathcaraaytayg |
| | tngtnwsngarggncarytngargaywsnwsnytnytngargtngayggnccngcnatgaara |
| | ncarwsngtntayggngtngaycaracncaywsnggnytnccnytncaycayathmgnmgn |
| | atgttyatgggngaywsntgyccntayytngcnggnaarccnaaratgttyttyathcaraaytayg |
| | tngtnwsngarggncarytngargaywsnwsnytnytngargtngayggncdngdnatgaara |
| | aygtngarttyaargdncaraarmgnggnytntgyadngtncaymgngargdngayttyttytgg |
| | wsnytntgyadngdngayatgwsnytnytngarcarwsncaywsnwsncdnwsnytntayyt |
| | ncartgyytnwsncaraarytnmgncargarmgnaarmgnccnytnytngayytncayathga |
| | rytnaayggntayatgtaygaytggaaywsnmgngtnwsngdnaargaraartaytaygtntgg |
| | ytncarcayacnytnmgnaaraarytnathytnwsntayacn (cFLIP Long “cFLIPL”) |
| 30 | atggcnacntaygargtnytntgygargtngcnmgnaarytnggnacngaygaymgngargtn |
| | gtnytnttyytnytnaaygtnttyathccncarccnacnytngcncarytnathggngcnytnmgn |
| | gcnytnaargargarggnmgnytnacnttyccnytnytngcngartgyytnttymgngcnggn |
| | mgnmgngayytnytnmgngayytnytncayytngayccnmgnttyytngarmgncayytn |
| | gcnggnacnatgwsntayttywsnccntaycarytnacngtnytncaygtngayggngarytnt |
| | gygcnmgngayathmgnwsnytnathttyytnwsnaargayacnathggnwsnmgnwsna |
| | cnccncaracnttyytncaytgggtntaytgyatggaraayytngayytnytnggnccnacngay |
| | gtngaygcnytnatgwsnatgytnmgnwsnytnwsnmgngtngayytncarmgncargtnc |
| | aracnytnatgggnytncayytnwsnggnccnwsncaywsncarcaytaymgncayacncc |
| | n (KSHV vFLIP) |
| 31 | atggayytnaaracnacngtnytncayathacngaywsnttyacngargargaratgtaytgyytn |
| | ytnttyytnathaayggntgyathccnmgnaaytgyaaygcngtnaarathwsngayytnathat |
| | hgaracnytnwsnaarwsnacncartgggayathtgyytnacncartgyytntaygtnytnmgn |
| | aarathgarytnytnytnaayytnttycargtnacnaargargaygtnaarcarwsnttyttyacnca |
| | rytncarytngaracncaygtnytnacnytngtnaaygtnaayaayaayytnacngcnaargayg |
| | araarmgnytntgyttyathytngaycarttyttyccnmgnaaygtngtngcnwsnwsngtnath |
| | ytntgygtnttywsnaayatgytntgygaratgccngtnytngartgyytntgycarytnaaraartg |
| | yytnaarcarathggnmgnwsngayytngcnaaracngtn (HVS vFLIP) |
| 32 | atgwsngaywsnaargargtnccnwsnytnccnttyytnmgncayytnytngargarytngay |
| | wsncaygargaywsnytnytnytnttyytntgycaygaygcngcnccnggntgyacnacngtn |
| | acncargcnytntgywsnytnwsncarcarmgnaarytnacnytngcngcnytngtngaratgy |
| | tntaygtnytncarmgnatggayytnytnaarwsnmgnttyggnytnwsnaargarggngcng |
| | arcarytnytnggnacnwsnttyytnacnmgntaymgnaarytnatggtntgygtnggngarga |
| | rytngaywsnwsngarytnmgngcnytnmgnytnttygcntgyaayytnaayccnwsnytn |
| | wsnacngcnytnwsngarwsnwsnmgnttygtngarytngtnytngcnytngaraaygtngg |
| | nytngtnwsnccnwsnwsngtnwsngtnytngcngayatgytnmgnacnytnmgnmgnyt |
| | ngayytntgycarcarytngtngartaygarcarcargarcargcnmgntaymgntaytgytayg |
| | cngcnwsnccnwsnytnccngtnmgnacnytnmgnmgnggncayggngcnwsngarca |
| | ygarcarytntgyatgccngtncargarwsnwsngaywsnccngarytnytnmgnacnccngt |
| | ncargarwsnwsnwsngaywsnccngarcaracnacn (MCV vFLIP) |
| 33 | atgwsngcngargtnathcaycargtngargargcnytngayacngaygaraargaratgytnyt |
| | nttyytntgymgngaygtngcnathgaygtngtnccnccnaaygtnmgngayytnytngayat |
| | hytnmgngarmgnggnaarytnwsngtnggngayytngcngarytnytntaymgngtnmg |
| | nmgnttygayytnytnaarmgnathytnaaratggaymgnaargcngtngaracncayytnyt |
| | nmgnaayccncayytngtnwsngaytaymgngtnytnatggcngarathggngargayytng |
| | ayaarwsngaygtnwsnwsnytnathttyytnatgaargaytayatgggnmgnggnaarathw |
| | snaargaraarwsnttyytngayytngtngtngarytngaraarytnaayytngtngcnccngayc |
| | arytngayytnytngaraartgyytnaaraayathcaymgnathgayytnaaracnaarathcara |
| | artayaarcarwsngtncarggngcnggnacnwsntaymgnaaygtnytncargcngcnathc |
| | araarwsnytnaargayccnwsnaayaayttymgnatgathacnccntaygcncaytgyccng |
| | ayytnaarathytnggnaaytgywsnatg (cFLIP Short “cFLIPs”) |
| 34 | mgnmgnmgncarmgnmgnaaraarmgnggntayggn (TAT-DOMAIN) |
| 35 | mgnmgnmgncarmgnmgnaaraarmgnggntayggnttygtnaayytnytnttyytngtng |
| | tngar (TAT-α2) |
| 36 | mgnmgnmgncarmgnmgnaaraarmgnggntayggnttygtnaayytngcngcngcngt |
| | ngtngar (TAT-α2m) |
| 37 | mgnmgnmgncarmgnmgnaaraarmgnggntayggnaaygaratgtgytaygtntggcay |
| | ytnttyacncar (TAT-α2) |
| 38 | mgnmgnmgncarmgnmgnaaraarmgnggntayggnaaygaratgtgygcngcngcnca |
| | ygcngcnacncar (TAT-α4M) |
| 39 | athytncaracnmgnacntaygayytntayathacntaygayaartaytaycaracnccnmgnyt |
| | ntggytnttyggntaygaygarcarmgncarccnytnacngtngarcayatgtaygargayathw |
| | sncargaycaygtnaaraaracngtnacnathgaraaycayccncayytnccnccnccnccnatg |
| | tgywsngtncayccntgymgncaygcngargtn (Atg 3 Binding Domain-SEQ |
| | ID NO.: 19) |
| 40 | gtnatgaaraarathathgaracngtngcngarggnggnggngarytnggngtncayatgtayyt |
| | nytnathttyytnaarttygtncargcngtnathccnacnathgartaygaytayacnmgncaytty |
| | acnatg (Atg 3 Binding Domain-SEQ ID NO.: 20) |

The nucleotide symbols of Table 5 indicate the following:
a = Adenine;
c = Cytosine;
g = Guanine;
t = Thymine;
u = Uracil;
r = Guanine/Adenine;
y = Cytosine/Thymine;
k = Guanine/Thymine;
m = Adenine/Cytosine;
s = Guanine/Cytosine;
w = Adenine/Thymine;
b = Guanine/Thymine/Cytosine;
d = Guanine/Adenine/Thymine;
h = Adenine/Cytosine/Thymine;
v = Guanine/Cytosine/Adenine;
n = Adenine/Guanine/Cytosine/Thymine.

Antibodies that bind to the proteins, polypeptides, variants, hybrids, and/or peptide fragments and biological equivalents of each thereof, described above are further provided by this invention. The proteins, peptide fragments, hybrids, and equivalents of each thereof, and peptide fragment compositions also are useful to raise antibodies that in turn have commercial, diagnostic and/or therapeutic utility. The antibodies are provided alone or in combination with a carrier such as a pharmaceutically acceptable carrier for therapeutic or diagnostic application. Portions of these antibodies are also provided that include, but are not limited to, an intact antibody molecule, a single chain variable region (ScFv), a monoclonal antibody, a polyclonal antibody, a chimeric antibody, a humanized antibody, a veneered antibody or a human antibody. Methods to raise antibodies are also provided herein. The antibodies can be generated in any appropriate in vitro or in vivo system, e.g., in cell culture, in phage, or in various animals, including but not limited to cows, rabbits, goats, mice, rats, hamsters, guinea pigs, sheep, dogs, cats, monkeys, chimpanzees, apes, etc., using techniques known in the art such as Multiple Antigenic Peptides, described herein.

This invention also provides compositions containing one or more of: an isolated protein, an isolated polynucleotide, an isolated peptide fragment; isolated variant, isolated hybrid, or a biological equivalent of each thereof, an antibody, an isolated peptide, an antibody fragment, variant or derivative, a host cell containing one or more of these compositions, in combination of any of the above, with a carrier, e.g., a pharmaceutically acceptable carrier or a solid phase carrier such as a chip or array support. The compositions can be used in methods to inhibit cancer cell growth, increase or induce cancer cell death, diminish or treat or ameliorate cancer or solid malignant tumors, eliminate viral particles associated with a viral infection, and/or treat or ameliorate neurodegenerative diseases such as Alzheimer's disease, Parkinson's disease, Huntington's disease, and neuronal degeneration. Alternatively, they can be used for screening for small molecules or other agents that mimic the activity or function of the polypeptides, peptide fragments, variant, hybrid, or an equivalent of each thereof, antibodies and/or polynucleotides.

The proteins, polynucleotides, polypeptides, antibodies and compositions of the present invention also can be used in the manufacture of medicaments for the treatment of humans and other animals by administration in accordance with conventional procedures, such as an active ingredient in pharmaceutical compositions. Accordingly, compositions containing the proteins, polynucleotides, polypeptides, antibodies as described herein are further provided with a solid or liquid phase carrier such as a pharmaceutically acceptable carrier.

### BRIEF DESCRIPTION OF THE DRAWINGS

FIG. 1 A-G show that the KSHV-α4 and HVS-α4 induced cell deaths in various ALL cells at 30 μM and 50 μM concentration.

FIG. 2 demonstrates that HVS-α4 induced cell death occurred at least as early as half an hour (A) and was dose dependent (B).

FIG. 3 shows the sequences (A) of variants and hybrids of KSHV-α4 (Kα4) and HVS-α4 (H-α4) and their anticancer activities (B).

FIG. 4 shows the anti-cancer activity of SMER (small molecule enhancer of rapamycin) as disclosed in Sarkar et al. (2007) Nature Chemical Biology 3:331, in ALL cells, in RCH-AcV (A) and in SUP-B15 (B).

FIG. 5 compares the anticancer activities among KSHV-α2, α4 and α4M in ALL cell lines BV-173(A) and SUP-B15 (B).

FIG. 6 shows KSHV-α4 and HVS-α4 induced cell death in human T cell lymphoblast-like cells (A) and human erythromyeloblastoid leukemia cells (B).

FIG. 7 shows the anticancer activities of cellular-α4, HVS-α4, KSHV-α4 in LNCap cells, a prostate cancer line (A), 293T cells (B) and HCT116, a colon cancer cell line (C).

FIG. 8 shows that cell count (A) and relative luminescence chart (B) the anticancer activities of cellular-α4, HVS-α4 and KSHV-α4 in LNCaP cells.

### DETAILED DESCRIPTION OF THE INVENTION

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

### DEFINITIONS

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state as well as substantially purified from an extract thereof. The term “isolated” is also used herein to refer to polypeptides, antibodies, proteins, host cells and polynucleotides that are isolated from other cellular proteins or tissues and is meant to encompass both purified and recombinant polypeptides, antibodies, proteins and polynucleotides. In other embodiments, the term “isolated” means separated from constituents, cellular and otherwise, in which the cell, tissue, polynucleotide, peptide, polypeptide, protein, antibody or fragment(s) thereof, which are normally associated in nature and can include at least 80%, or alternatively at least 85%, or alternatively at least 90%, or alternatively at least 95%, or alternatively at least 98%, purified from a cell or cellular extract. For example, an isolated polynucleotide is separated from the 3′ and 5′ contiguous nucleotides with which it is normally associated in its native or natural environment, e.g., on the chromosome. An isolated cell, for example, is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “binding” or “binds” as used herein are meant to include interactions between molecules that may be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “FLIP” is conventionally defined as a FLICE-like inhibitor protein having two death effector domains, DED1 and DED2. (Thome and Tschopp (2001) Nat. Rev. Immunol. 1:50-58). As used herein, the term “cFLIP” refers to the short and long form of cellular FLIP. cFLIPs refers to the short form of cFLIP. cFLIP_{L} refers to the long form of cFLIP. The “viral” form of FLICE-like inhibitor protein refers to viral FLIP (vFLIP) any one of Kaposi's sarcoma-associated herpesvirus (KSHV), Herpesvirus saimiri (HVS), or Molluscum contagiosum virus (MCV). As used herein, “FLIP” refers cFLIP or vFLIP.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment” as used herein, also refers to a peptide chain.

A “hybrid peptide” between a first peptide and one or more other peptides (collectively referred to as the “original peptides”) refers to a peptide having at least one amino acid identical to that of another peptide at the same position. In one aspect, a hybrid peptide includes fewer than about 60% amino acids from any of the original peptides. In one aspect, a hybrid peptide is a fusion peptide between a fragment of the first original peptide and a fragment of one of the other original peptides. An example of such is SEQ ID NO. 42. In a particular aspect, the fragment is an N-terminal or a C-terminal fragment. In another aspect, a hybrid peptide is not homologous to any of the original peptides. A “hybrid peptide” of a peptide, such as an α4 FLIP peptide fragment, refers to a hybrid peptide between the peptide and one or more peptides of the same kind, such as other a4 FLIP peptide fragments.

The phrase “biologically equivalent polypeptide” or “biologically equivalent peptide or peptide fragment” or “biologically equivalent polynucleotide” refers to a protein or a peptide fragment which is homologous to the exemplified protein or peptide fragment and which exhibit similar biological activity in vitro or in vivo, e.g., approximately 100%, or alternatively, over 90% or alternatively over 85% or alternatively over 70%, as compared to the standard or control biological activity. Additional embodiments within the scope of this invention are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence identity or homology. Percentage homology can be determined by sequence comparison using programs such as BLAST run under appropriate conditions. In one aspect, the program is run under default parameters.

As used herein, a variant is a peptide selected from SEQ ID NOS. 2, 4, 6 or 8, having a substitution with 1, 2, 3, or 4 amino acids at the corresponding positions of the a region of one or more other peptides selected from SEQ ID NOS. 2, 4, 6 or 8.

As understood by those of skill in the art, a “retro-inverso” refers to an isomer of a linear peptide in which the direction of the sequence is reversed (“retro”) and the chirality of each amino acid residue is inverted (“inverso”). Compared to the parent peptide, a helical retro-inverso peptide can substantially retain the original spatial conformation of the side chains but has reversed peptide bonds, resulting in a retro-inverso isomer with a topology that closely resembles the parent peptide, since all peptide backbone hydrogen bond interactions are involved in maintaining the helical structure. See Jameson et al., (1994) Nature 368:744-746 (1994) and Brady et al. (1994) Nature 368:692-693. The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into an D retro-inverso peptide by synthesizing a reverse of the sequence for the corresponding native L-amino acid sequence.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “non-contiguous” refers to the presence of an intervening peptide, nucleotide, polypeptide or polynucleotide between a specified region and/or sequence. For example, two polypeptide sequences are non-contiguous because the two sequences are separated by a polypeptide sequences that is not homologous to either of the two sequences. Non-limiting intervening sequences are comprised of at least a single amino acid or nucleotide.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

“Polycistronic” refers to a form of gene organization that results in transcription of an mRNA that codes for multiple gene products, each of which is independently translated from the mRNA.

A “gene product” or alternatively a “gene expression product” refers to the RNA when a gene is transcribed or amino acid (e.g., peptide or polypeptide) generated when a gene is transcribed and translated.

“Short interfering RNA” (siRNA) refers to sequence-specific or gene specific suppression of gene expression (protein synthesis) that is mediated by double-stranded RNA molecules, generally, from about 10 to about 30 nucleotides long that are capable of mediating RNA interference (RNAi). As used herein, the term siRNA includes short hairpin RNAs (shRNAs).

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge.

A “gene delivery vehicle” is defined as any molecule that can carry inserted polynucleotides into a host cell. Examples of gene delivery vehicles are liposomes, micelles, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

The terms “culture” or “culturing” refer to the in vitro propagation of cells or organisms on or in media of various kinds It is understood that the descendants of a cell grown in culture may not be completely identical (i.e., morphologically, genetically, or phenotypically) to the parent cell.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are know in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarrying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers homologous to a polynucleotide, polypeptide or antibody described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the gene(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

Other non-limiting examples of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding protein, peptide, antibody or antigen, or will be able to ascertain the same by use of routine experimentation.

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Cell,” “host cell” or “recombinant host cell” are terms used interchangeably herein. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

The terms “disease” and “disorder” are used inclusively and refer to any condition associated with regulation of autophagy. In the context of this invention the disease may be associated with cancer, a neurodegenerative disorder, or a pathogenic infection. As used herein, “cancer” may refer both to precancerous cells as well as cancerous cells of a tumor such as a solid tumor. Examples of neurodegenerative diseases include, but are not limited to, Alzheimer's disease, Parkinson's disease, Huntington's disease, and transmissible spongiform encephalopathies. Examples of pathogenic infections include, but are not limited to, infection by bacteria such as group A Streptococcus, Mycobacterium tuberculosis, Shigella flexneri, Salmonella enterica, Listeria monocytogenes, Francisella tularensis, and infection by viruses such as herpes simplex virus.

“Treating,” “treatment,” or “ameliorating” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are know to be predisposed to the disease due to family history, being genetically predispose to developing the disease, or diagnosed with a disease or disorder that predisposes them to developing the disease to be treated.

### Descriptive Embodiments

### Isolated Peptide Fragments and Compositions

This invention provides isolated peptide fragments, hybrids and variants of these fragments and biological equivalent of each thereof, of vFLIP and cFLIP proteins that inhibit or diminish the ability of cFLIP or vFLIP to bind to Atg3 and inhibit formation of the LC3-Atg4-Atg7-Atg3 conjugation complex that is necessary for autophagy induction. The peptide fragments are useful therapeutically to augment or promote autophagy in a cell, tissue or subject and can be administered at different stages of cell growth to promote autophagy of the cell. In one aspect, they will modify autophagy and increase cell death. So in one application, the polypeptides inhibit the growth of precancerous cells, malignant tumors and cancer cells, increase or induce cancer cell death, eliminate viral particles associated with a viral infection, and/or treat or ameliorate neurodegenerative diseases by inducing or increasing autophagy. In a further aspect, the compositions are administered to a precancerous cell that has or will be subsequently treated with a chemotherapeutic agent.

Thus, in one aspect this invention provides an isolated peptide fragment comprising, or alternatively consisting essentially of, or yet further consisting of, a region of the vFLIP or cFLIP protein that binds to Atg3, or a portion thereof. Examples of these fragments are identified in SEQ ID NOS. 1 through 8, 15 through 18 and 41 through 47 including variants and hybrids of each thereof, as well as biological equivalents of each thereof. In some aspects, this invention provides an isolated peptide fragment comprising, or alternatively consisting essentially of, or yet further consisting of, a region of the death effector domain (DED) of vFLIP or cFLIP, or a portion thereof. See Mol. Cell. (2008) 30:262. In another aspect, this invention provides an isolated peptide fragment comprising, or alternatively consisting essentially of, or yet further consisting of, an alpha-helix region of a DED of vFLIP or cFLIP, or a portion thereof. In another aspect, this invention provides an isolated peptide fragment comprising, or alternatively consisting essentially of, or yet further consisting of, an alpha-helix region of a DED of vFLIP or cFLIP, or a portion thereof. Non-limiting examples are show in SEQ ID NO. 10, wherein the DED1 of vFLIP is from amino acid 1 to amino acid 90 and the DED2 of vFLIP is from amino acid 91 to 188.

In another aspect this invention provides an isolated peptide fragment of vFLIP comprising, or alternatively consisting essentially of, or yet further consisting of, the amino acid sequence EVVLFLLNVF (SEQ ID NO. 1) or a peptide fragment substantially homologous and biologically equivalent to SEQ ID NO. 1, a variant or hybrid thereof, or alternatively the retro-inverso form or biological equivalent of each thereof. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 1, each as determined using methods known to those skilled in the art and identified herein, when run under default parameters. Preferred amino acid substitutions for the biologically equivalent polypeptides of SEQ ID NO. 1 are described infra.

In still another aspect this invention provides an isolated peptide fragment of vFLIP comprising, or alternatively consisting essentially of, or yet further consisting of, the amino acid sequence QTFLHWVYCMEN (SEQ ID NO. 2) or a peptide fragment substantially homologous and biologically equivalent to SEQ ID NO. 2 a variant or hybrid thereof, or alternatively the retro-inverso form or a biological equivalent of each thereof, of these peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 2, each as determined using methods known to those skilled in the art and identified herein, when run under default parameters. Preferred amino acid substitutions for the biologically equivalent polypeptides of SEQ ID NO. 2 are described infra.

In another aspect, provided herein is a variant of SEQ ID NO. 2 that includes substitution with one or more amino acids at the corresponding positions of the α region of another FLIP peptide, such as but not limited to, SEQ ID NOS. 4, 6 or 8. In one aspect, the substitution with one or more amino acids is substitution with 1, or alternatively 2, or 3, or 4, or 5, or 6 amino acids from SEQ ID NO. 4, or alternatively 6, or alternatively 8, individually or in combination. In some embodiments, disclosed and provided is a hybrid peptide between SEQ ID NO. 2 and any one or more of SEQ ID NOS. 4, 6 or 8. Examples of such are shown in Table 6.

TABLE 6

| Name | SEQ ID NO | Sequence |
|---|---|---|
| M1 | 41 | SSVILCVYCMEN |
| M2 | 42 | QTFLHWVFSNML |
| M3 | 43 | QTFLLWVYCMEN |
| M4 | 44 | QTFLHCVYCMEN |
| M5 | 45 | QTFLLCVYCMEN |
| M6 | 46 | QTFLHWVYCMMN |
| M7 | 47 | QTFLLCVYCMMN |

TABLE 7

| SEQ | | |
|---|---|---|
| ID | | Encoded |
| NO. | Nucleic Acid Consensus Sequence | peptide |
| 48 | wsnwsngtnathytntgygtntaytgyatggaraay | M1 |
| 49 | caracnttyytncaytgggtnttywsnaayatgytn | M2 |
| 50 | caracnttyytnytntgggtntaytgyatggaraay | M3 |
| 51 | caracnttyytncaytgygtntaytgyatggaraay | M4 |
| 52 | caracnttyytnytntgygtntaytgyatggaraay | M5 |
| 53 | caracnttyytncaytgggtntaytgyatgatgaay | M6 |
| 54 | caracnttyytnytntgygtntaytgyatgatgaay | M7 |

Unexpectedly and referring to Table 6, Applicants have found that modification of the amino acid 5 from H to L and modification of amino acid 6 from C to W substantially increased cell killing activity. Thus, biological equivalents of these peptides, as provided herein, should retain at least L at amino acid position 5, or W at amino acid position 6, or each of L at position 5 and W at amino acid 6. In a further aspect, modification of amino acid 11 to M also enhanced autophagy. This further modification can be independent or combined with either one or both modifications at amino acids 5 and 6. The other amino acids can further be modified.

Thus, in another aspect, provided is an isolated polypeptide comprising, or alternatively consisting essentially of, or yet further consisting of, the amino acid sequence SSVILCVYCMEN (SEQ ID NO. 41) or a variant or hybrid or a peptide substantially homologous and biologically equivalent to SEQ ID NO. 41 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 41, each as determined using methods known to those skilled in the art and identified herein, when run under default parameters. Preferred amino acid substitutions for the biologically equivalent polypeptides of SEQ ID NO. 41 are described supra.

In another aspect this invention provides an isolated polypeptide comprising, or alternatively consisting essentially of, or yet further consisting of, the amino acid sequence QTFLHWVFSNML (SEQ ID NO. 42) or a variant or hybrid or a polypeptide substantially homologous and biologically equivalent to SEQ ID NO. 42 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 42, each as determined using methods known to those skilled in the art and identified herein, when run under default parameters. Preferred amino acid substitutions for the biologically equivalent polypeptides of SEQ ID NO. 42 are described supra.

In another aspect this invention provides an isolated polypeptide fragment comprising, or alternatively consisting essentially of, or yet further consisting of, the amino acid sequence QTFLLWVYCMEN (SEQ ID NO. 43) or a variant or hybrid or a peptide substantially homologous and biologically equivalent to SEQ ID NO. 43 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 43, each as determined using methods known to those skilled in the art and identified herein, when run under default parameters. Preferred amino acid substitutions for the biologically equivalent polypeptides of SEQ ID NO. 43 are described supra.

In another aspect, provided is an isolated polypeptide comprising, or alternatively consisting essentially of, or yet further consisting of, the amino acid sequence QTFLHCVYCMEN (SEQ ID NO. 44) or a variant or hybrid or a peptide substantially homologous and biologically equivalent to SEQ ID NO. 44 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 44, each as determined using methods known to those skilled in the art and identified herein, when run under default parameters. Preferred amino acid substitutions for the biologically equivalent polypeptides of SEQ ID NO. 44 are described supra.

In another aspect this invention provides an isolated polypeptide comprising, or alternatively consisting essentially of, or yet further consisting of, the amino acid sequence QTFLLCVYCMEN (SEQ ID NO. 45) or a variant or hybrid or a peptide substantially homologous and biologically equivalent to SEQ ID NO. 45 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 45, each as determined using methods known to those skilled in the art and identified herein, when run under default parameters. Preferred amino acid substitutions for the biologically equivalent polypeptides of SEQ ID NO. 45 are described supra.

In another aspect this invention provides an isolated peptide comprising, or alternatively consisting essentially of, or yet further consisting of, the amino acid sequence QTFLHWVYCMMN (SEQ ID NO. 46) or a variant or hybrid or a peptide substantially homologous and biologically equivalent to SEQ ID NO. 46 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 46, each as determined using methods known to those skilled in the art and identified herein, when run under default parameters. Preferred amino acid substitutions for the biologically equivalent polypeptides of SEQ ID NO. 46 are described supra.

In another aspect this invention provides an isolated peptide comprising, or alternatively consisting essentially of, or yet further consisting of, the amino acid sequence QTFLLCVYCMMN (SEQ ID NO. 47) or a variant or hybrid or a peptide substantially homologous and biologically equivalent to SEQ ID NO. 47 or alternatively the retro-inverso form or a biological equivalent of each thereof. Substantially homologous and biologically equivalent peptides intend those having at least 80% homology, or alternatively at least 85% homology, or alternatively at least 90% homology, or alternatively, at least 95% homology or alternatively, at least 98% homology to SEQ ID NO. 47, each as determined using methods known to those skilled in the art and identified herein, when run under default parameters. Preferred amino acid substitutions for the biologically equivalent polypeptides of SEQ ID NO. 47 are described infra.

In yet another aspect this invention provides an isolated peptide fragment of cFLIP comprising, or alternatively consisting essentially of, or yet further consisting of, the amino acid sequence EMLLFLCRDV (SEQ ID NO. 3) or a variant or hybrid or a peptide fragment substantially homologous and biologically equivalent to SEQ ID NO. 3 or alternatively, the retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 3, each as determined using methods known to those skilled in the art and identified herein, when run under default parameters. Preferred amino acid substitutions for the biologically equivalent polypeptides of SEQ ID NO. 3 are described infra.

In still another aspect this invention provides an isolated peptide fragment of cFLIP comprising, or alternatively consisting essentially of, or yet further consisting of, the amino acid sequence KSFLDLVVELEK (SEQ ID NO. 4) or a variant or hybrid or a peptide fragment substantially homologous and biologically equivalent to SEQ ID NO. 4 or alternatively the retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 4, each as determined using methods known to those skilled in the art and identified herein, when run under default parameters. Preferred amino acid substitutions for the biologically equivalent polypeptides of SEQ ID NO. 4 are described infra.

Also provided is a variant of SEQ ID NO. 4 that includes substitution with one or more amino acids at the corresponding positions of the a region of another FLIP peptide, such as but not limited to, SEQ ID NOS. 2, 6 or 8. In one aspect, the substitution with one or more amino acids is substitution with 1, or alternatively 2, or 3, or 4, or 5, or 6 amino acids from SEQ ID NO. 2, or alternatively 6, or alternatively 8, individually or in combination. In some embodiments, the variant of SEQ ID NO. 4 is a hybrid peptide between SEQ ID NO. 4 and any one or more of SEQ ID NOS. 2, 6 or 8.

In another aspect this invention provides an isolated peptide fragment of HVS vFLIP comprising, or alternatively consisting essentially of, or yet further consisting of, the amino acid sequence YCLLFLINGC (SEQ ID NO. 5) or a variant or hybrid or a peptide fragment substantially homologous and biologically equivalent to SEQ ID NO. 5 or alternatively the retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 5, each as determined using methods known to those skilled in the art and identified herein, when run under default parameters. Preferred amino acid substitutions for the biologically equivalent polypeptides of SEQ ID NO. 5 are described infra.

In another aspect this invention provides an isolated peptide fragment of HVS vFLIP comprising, or alternatively consisting essentially of, or yet further consisting of, the amino acid sequence SSVILCVFSNML (SEQ ID NO. 6) or a variant or hybrid or a peptide fragment substantially homologous and biologically equivalent to SEQ ID NO. 6, or alternatively, the retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 6, each as determined using methods known to those skilled in the art and identified herein, when run under default parameters. Preferred amino acid substitutions for the biologically equivalent polypeptides of SEQ ID NO. 6 are described infra.

Further provided is a variant of SEQ ID NO. 6 that includes substitution with one or more amino acids at the corresponding positions of the a region of another FLIP peptide, such as but not limited to, SEQ ID NOS. 2, 4 or 8. In one aspect, the substitution with one or more amino acids is substitution with 1, or alternatively 2, or 3, or 4, or 5, or 6 amino acids from SEQ ID NO. 2, or alternatively 4, or alternatively 8, individually or in combination. In some embodiments, the variant of SEQ ID NO. 6 is a hybrid peptide between SEQ ID NO. 6 and any one or more of SEQ ID NOS. 2, 4 or 8.

In another aspect this invention provides an isolated peptide fragment of MCV MC159 comprising, or alternatively consisting essentially of, or yet further consisting of, the amino acid sequence SLLLFLCHDA (SEQ ID NO. 7) or a variant or hybrid or a peptide fragment substantially homologous and biologically equivalent to SEQ ID NO. 7 or alternatively, the retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 7, each as determined using methods known to those skilled in the art and identified herein, when run under default parameters. Preferred amino acid substitutions for the biologically equivalent polypeptides of SEQ ID NO. 7 are described infra.

In another aspect this invention provides an isolated peptide fragment of MCV MC159 comprising, or alternatively consisting essentially of, or yet further consisting of, the amino acid sequence SRFVELVLALEN (SEQ ID NO. 8) or a variant or hybrid or a peptide fragment substantially homologous and biologically equivalent to SEQ ID NO. 8 or alternatively, a retro-inverso forms or a biological equivalent of each thereof of the peptides. Substantially homologous and biologically equivalent peptide fragments intend those having at least 80% homology, or alternatively at least 85% homology, or alternatively at least 90% homology, or alternatively, at least 95% homology or alternatively, at least 98% homology to SEQ ID NO. 8, each as determined using methods known to those skilled in the art and identified herein, when run under default parameters. Preferred amino acid substitutions for the biologically equivalent polypeptides of SEQ ID NO. 8 are described infra.

Further provided is a variant of SEQ ID NO. 8 that includes substitution with one or more amino acids at the corresponding positions of the a region of another FLIP peptide, such as but not limited to, SEQ ID NOS. 2, 4 or 6. In one aspect, the substitution with one or more amino acids is substitution with 1, or alternatively 2, or 3, or 4, or 5, or 6 amino acids from SEQ ID NO. 2, or alternatively 4, or alternatively 6, individually or in combination. In some embodiments, a biological equivalent of SEQ ID NO. 8 is a hybrid peptide between SEQ ID NO. 8 and any one or more of SEQ ID NOS. 2, 4 or 6.

Another aspect of this invention is an isolated peptide fragment comprising, or alternatively consisting essentially of, or yet further consisting of, two non-contiguous death effector domain regions of cFLIP, wherein the regions comprise the amino acid sequences EVVLFLLNVF (SEQ ID NO. 1) and QTFLHWVYCMEN (SEQ ID NO. 2), or amino acid sequences substantially homologous and biologically equivalent to these polypeptides. Substantially homologous and biologically equivalent polypeptides intend polypeptides having at least 60%, or alternatively at least 65% homology, or alternatively at least 70% homology, or alternatively at least 75% homology, or alternatively at least 85% homology, or alternatively at least 90% homology, or alternatively, at least 95% homology or alternatively, at least 98% homology to SEQ ID NOS. 1 and 2, each as determined using methods known to those skilled in the art and identified herein, when run under default parameters. Preferred amino acid substitutions for the biologically equivalent peptides are described infra. Examples of substantially homologous and biologically equivalent to SEQ ID NO. 2, without limitation, are shown in Table 6. Also within the scope of this invention are the retro-inverso forms of these peptides.

Another aspect of this invention is an isolated peptide fragment comprising, or alternatively consisting essentially of, or yet further consisting of, two non-contiguous death effector domain regions of vFLIP, wherein the regions comprise the amino acid sequences EMLLFLCRDV (SEQ ID NO. 3) and KSFLDLVVELEK (SEQ ID NO. 4), or amino acid sequences substantially homologous and biologically equivalent to these polypeptides. Substantially homologous and biologically equivalent polypeptides intend polypeptides having at least 60%, or alternatively at least 65% homology, or alternatively at least 70% homology, or alternatively at least 75% homology, or alternatively at least 85% homology, or alternatively at least 90% homology, or alternatively, at least 95% homology or alternatively, at least 98% homology to SEQ ID NOS. 3 and 4, each as determined using methods known to those skilled in the art and identified herein, when run under default parameters. Preferred amino acid substitutions for the biologically equivalent peptides are described infra. Also within the scope of this invention are the retro-inverso forms of these peptides.

Another aspect of this invention is an isolated peptide fragment comprising, or alternatively consisting essentially of, or yet further consisting of, two non-contiguous death effector domain regions of HVS vFLIP, wherein the regions comprise the amino acid sequences YCLLFLINGC (SEQ ID NO. 5) and SSVILCVFSNML (SEQ ID NO. 6), or amino acid sequences substantially homologous and biologically equivalent to these polypeptides. Substantially homologous and biologically equivalent polypeptides intend polypeptides having at least 60%, or alternatively at least 65% homology, or alternatively at least 70% homology, or alternatively at least 75% homology, or alternatively at least 85% homology, or alternatively at least 90% homology, or alternatively, at least 95% homology or alternatively, at least 98% homology to SEQ ID NOS. 5 and 6, each as determined using methods known to those skilled in the art and identified herein, when run under default parameters. Preferred amino acid substitutions for the biologically equivalent peptides are described infra. Also within the scope of this invention are the retro-inverso forms of these peptides.

Another aspect of this invention is an isolated peptide fragment comprising, or alternatively consisting essentially of, or yet further consisting of, two non-contiguous death effector domain regions of MCV MC159, wherein the regions comprise the amino acid sequences SLLLFLCHDA (SEQ ID NO. 7) and SRFVELVLALEN (SEQ ID NO. 8), or amino acid sequences substantially homologous and biologically equivalent to these polypeptides. Substantially homologous and biologically equivalent polypeptides intend polypeptides having at least 60%, or alternatively at least 65% homology, or alternatively at least 70% homology, or alternatively at least 75% homology, or alternatively at least 85% homology, or alternatively at least 90% homology, or alternatively, at least 95% homology or alternatively, at least 98% homology to SEQ ID NOS. 7 and 8, each as determined using methods known to those skilled in the art and identified herein, when run under default parameters. Preferred amino acid substitutions for the biologically equivalent peptides are described infra. Also within the scope of this invention are the retro-inverso forms of these peptides.

Further provided by this invention is an isolated peptide that comprises, or alternatively consisting essentially of, or yet further consisting of, a plurality of polypeptides having two or more non-contiguous amino acid sequences of the group: (SEQ ID NO. 1); (SEQ ID NO. 2); (SEQ ID NO. 3); (SEQ ID NO. 4); (SEQ ID NO. 5); (SEQ ID NO. 6); (SEQ ID NO. 7); (SEQ ID NO. 41); (SEQ ID NO. 42); (SEQ ID NO. 43;) (SEQ ID NO. 44); (SEQ ID NO. 45); (SEQ ID NO. 46); and (SEQ ID NO. 47) and/or or a variant or hybrid or their biological equivalents and/or the retro-inverso forms of each, examples of some which are identified in Table 3.

In one aspect, the fragment contains at least one of SEQ ID NO. 1, 3, 5, 7, 41, 42, 43, 44, 45, 46, or 47 or alternatively, at least one of SEQ ID NOS. 2, 4, 6 or 8, or a variant or hybrid containing it or their biological equivalents and/or retro-inverso forms thereof. In one specific aspect, the isolated peptide comprises, or alternatively consists essentially of, or yet further consists of, SEQ ID NOS. 1 and 2; or 3 and 4; or 5 and 6 or 7 and 8, or their biological equivalents or retro-inverso forms thereof.

Yet further provided is an isolated peptide fragment having one or more polypeptides having varying degrees of sequence identity or homology to one or more of SEQ ID NOS. 1 through 8 or 41 through 47 (with, in one aspect the further limitation that amino acid 5 and/or 6 is L and/or W, respectively), e.g., at least 65% homology, or alternatively at least 70% homology, or alternatively at least 75% homology, or alternatively at least 85% homology, or alternatively at least 90% homology, or alternatively, at least 95% homology or alternatively, at least 98% homology to SEQ ID NOS. 1 through 8 or 41 through 47 (with, in one aspect the further limitation that amino acid 5 and/or 6 is L or W, respectively), each as determined using methods known to those skilled in the art and identified herein, when run under default parameters.

Yet further provided is an isolated peptide fragment having one or more polypeptides having additional amino acids added onto the carboxyl-terminal end or amino-terminal end of the polypeptides of SEQ ID NOS. 1 through 8 and/or 41 through 47 such that the length of the peptide comprises an additional at least 10 amino acids, or alternatively at least 15 amino acids, or alternatively at least 20 amino acids, or alternatively at least 25 amino acids, or alternatively at least 30 amino acids, or alternatively at least 40 amino acids, or alternatively at least 50 amino acids, each amino acid added using methods known to those skilled in the art. Any of these larger peptide fragments which can in one aspect contain the contiguous amino acids as shown in the respective SEQ ID NOS. 9 through 13, be substituted in the appropriate compositions, host cells, vectors and methods as described herein. Similar to the smallest fragment shown in SEQ ID NOS. 1 through 8, this invention provides the retro-inverso form and biological equivalent forms of these larger peptide fragments. In one aspect, conjugated to the polypeptide is an amino acid that facilitates entry of the polypeptide into the cell such as the HIV TAT sequence.

It is known to those skilled in the art that modifications can be made to any peptide to provide it with altered properties. Peptide fragments of the invention can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobic, size, or charge. Methods used to determine the appropriate amino acid to be substituted and for which amino acid are know to one of skill in the art. Non-limiting examples include empirical substitution models as described by Layoff et al. (1978) In Atlas of Protein Sequence and Structure Vol. 5 suppl. 2 (ed. MR. Day off), pp. 345-352. National Biomedical Research Foundation, Washington D.C.; PAM matrices including Day off matrices (Layoff et al. (1978), supra, or JET matrices as described by Jones et al. (1992) Compute. Appl. Basic. 8:275-282 and Gannet et al. (1992) Science 256:1443-1145; the empirical model described by Adak and Hasegawa (1996) J. Mol. Evil. 42:459-468; the block substitution matrices (BLOSSOM) as described by Henrico and Henrico (1992) Proc. Natl. Acad. Sci. USA 89:10915-10919; Poisson models as described by Neil (1987) Molecular Evolutionary Genetics. Columbia University Press, New York.; and the Maximum Likelihood (ML) Method as described by Muller et al. (2002) Mol. Biol. Evil. 19:8-13.

Accordingly, in yet another aspect the isolated peptide fragment may comprise, or alternatively consisting essentially of, or yet further consisting of, a “biologically equivalent” or “biologically active” peptide fragment encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. For example, one or more of the valise, isoleucine, leucine, methionine, phenylalanine, or tryptophan residues of the hydrophobic core of an alpha helix of a death effector domain may be modified or substituted with another hydrophobic residue such as valine, isoleucine, leucine, methionine, phenylalanine, or tryptophan. In some embodiments, one or more of the valine, isoleucine, leucine, methionine, phenylalanine, or tryptophan residues of the amino acid sequences of SEQ ID NO. 1, SEQ ID NO. 2, SEQ ID NO. 3, SEQ ID NO. 4, SEQ ID NO. 5, SEQ ID NO. 6, SEQ ID NO. 7, or SEQ ID NO. 8, or SEQ ID NO. 15, or SEQ ID NO. 16, or SEQ ID NO. 17 or SEQ ID NO. 18, may be modified or substituted with another hydrophobic residue such as valine, isoleucine, leucine, methionine, phenylalanine, or tryptophan.

Proteins and peptide fragments comprising, or alternatively consisting essentially of, or yet further consisting of, the amino acid sequences of the invention can be prepared by expressing polynucleotides encoding the polypeptide sequences of this invention in an appropriate host cell. This can be accomplished by methods of recombinant DNA technology known to those skilled in the art. Accordingly, this invention also provides methods for recombinantly producing the polypeptides of this invention in a eukaryotic or prokaryotic host cell, which in one aspect is further isolated from the host cell. The proteins and peptide fragments of this invention also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

The protein and peptide fragments may be operatively linked to a transduction domain for facilitated cell entry. Protein transduction offers an alternative to gene therapy for the delivery of therapeutic proteins into target cells, and methods involving protein transduction are within the scope of the invention. Protein transduction is the internalization of proteins into a host cell from the external environment. The internalization process relies on a protein or peptide which is able to penetrate the cell membrane. To confer this ability on a normally non-transducing protein, the non-transducing protein can be fused to a transduction-mediating protein such as the antennapedia peptide, the HIV TAT protein transduction domain, or the herpes simplex virus VP22 protein. See Ford et al. (2001) Gene Ther.8:1-4. As such the polypeptides of the invention can, for example, include modifications that can increase such attributes as stability, half-life, ability to enter cells and aid in administration, e.g., in vivo administration of the polypeptides of the invention. For example, polypeptides of the invention can comprise, or alternatively consisting essentially of, or yet further consisting of, a protein transduction domain of the HIV TAT protein as described in Schwarze, et al. (1999) Science 285:1569-1572, and exemplified below.

In a further aspect, any of the proteins or peptides of this invention can be combined with a detectable label such as a dye for ease of detection.

This invention also provides pharmaceutical composition for in vitro and in vivo use comprising, or alternatively consisting essentially of, or yet further consisting of a therapeutically effective amount of the FLIP peptide fragment that causes at least about 75%, or alternatively at least about 80%, or alternatively at least about 85%, or alternatively at least about 90%, or alternatively at least about 95%, or alternatively at least about 99% effectiveness in the methods provided herein when applied in a molar concentration of less than about 10 micromolar, or alternatively less than about 9 micromolar, or alternatively less than about 8 micromolar, or alternatively less than about 7 micromolar, or alternatively less than about 6 micromolar, or alternatively less than about 5 micromolar, or alternatively less than about 4 micromolar, or alternatively less than about 3 micromolar, or alternatively less than about 2 micromolar, or alternatively less than about 1 micromolar, or alternatively less than about 0.5 micromolar, or alternatively less than about 0.25 micromolar concentration, as compared to a control that does not receive the composition. Comparative effectiveness can be determined by suitable in vitro or in vivo methods as known in the art and described herein.

This invention also provides compositions for in vitro and in vivo use comprising, or alternatively consisting essentially of, or yet further consisting of one or more of the isolated peptide fragments or a variant or hybrid or a biological equivalent of each thereof, described herein and a pharmaceutically acceptable carrier. In one aspect, the compositions are pharmaceutical formulations for use in the therapeutic methods of this invention. In a further aspect, the invention provides a pharmaceutical composition comprising, or alternatively consisting essentially of, or yet further consisting of, the isolated peptide fragment or a variant or hybrid or a biological equivalent of each thereof, in a concentration such that a therapeutically effective amount of the or pharmacological dose of the composition causes at least a 75%, or alternatively at least a 80%, or alternatively at least a 85%, or alternatively at least a 90%, or alternatively at least a 95% or alternatively at least a 97% reduction in viral infectivity when applied in a molar concentration of less than 1 micromolar, to a culture of responsive virus (e.g., influenza virus) virion, as compared to a control that does not receive the composition. In alternative aspects, the pharmacological dose of the composition when applied to the is in the range of about 150 nM to about 2 micromolar, or about 200 nM to about 2 micromolar, or about 250 nM to about 2 micromolar, or about 300 nM to about 2 micromolar, or about 400 nM to about 2 micromolar, or about 450 nm to about 2 micromolar, or about 500 nM to about 2 micromolar, or about 550 nM to about 2 micromolar, or about 600 nM to about 2 micromolar, or about 700 nM to about 2 micromolar, or about 800 nM to about 2 micromolar, or about 900 nM to about 2 micromolar, or about 1 micromolar to about 2 micromolar, or about 1.5 micromolar to about 2 micromolar, or about 50 nM to about 1 micromolar, or about 100 nM to about 1 micromolar, or about 150 nM to about 1 micromolar, or about 200 nM to about 1 micromolar, or about 250 nM to about 1 micromolar, or about 300 nM to about 1 micromolar, or about 400 nM to about 1 micromolar, or about 450 nm to about 1 micromolar, or about 500 nM to about 1 micromolar, or about 550 nM to about 1 micromolar, or about 600 nM to about 1 micromolar, or about 700 nM to about 1 micromolar, or about 800 nM to about 1 micromolar. Comparative effectiveness can be determined by suitable in vitro or in vivo methods as known in the art and described herein.

### Isolated Polynucleotides and Compositions

This invention also provides isolated polynucleotides encoding the polypeptides and peptide fragments described above as well as biological equivalents of each thereof. In one aspect the isolated polynucleotides encode peptide fragments comprising the sequences (SEQ ID NOS: 1 through 8 or 15 through 18 or 41 through 47) and their biological equivalents, variants or hybrids between any two of them. In another aspect, the polynucleotides or their biological equivalents, variants or hybrids are labeled with a detectable marker or label, such as a dye or radioisotope, for ease of detection.

This invention also provides the isolated complementary polynucleotides to the sequences identified above, their biological equivalents or their complements. Complementarity can be determined using traditional hybridization under conditions of moderate or high stringency. As used herein, the term polynucleotide intends DNA and RNA as well as modified nucleotides. For example, this invention also provides the anti-sense polynucleotide strand, e.g. antisense RNA or siRNA to these sequences or their complements. One can obtain an antisense RNA using the sequences that encode SEQ ID NOS: 1 through 8 or 41 through 47 using a methodology known to one of ordinary skill in the art wherein the degeneracy of the genetic code provides several polynucleotide sequences that encode the same polypeptide or the methodology described in Van der Krol, et al. (1988) BioTechniques 6:958. In another aspect, the polynucleotides or their biological equivalents are labeled with a detectable marker or label, such as a dye or radioisotope, for ease of detection.

Also provided are isolated polynucleotides encoding substantially homologous and biologically equivalent peptide fragments to the inventive peptide fragments. Substantially homologous and biologically equivalent intends those having varying degrees of homology, such as at least 65%, or alternatively, at least 70%, or alternatively, at least 75%, or alternatively at least 80%, or alternatively, at least 85%, or alternatively at least 90%, or alternatively, at least 95%, or alternatively at least 97% homologous as defined above and which encode peptides having the biological activity to bind Atg3 as described herein. It should be understood although not always explicitly stated that embodiments to substantially homologous peptide fragments and polynucleotides are intended for each aspect of this invention, e.g., peptides, polynucleotides and antibodies.

The polynucleotides of this invention can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this invention also provides a process for obtaining the peptide fragments of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

In one aspect, the RNA is short interfering RNA, also known as siRNA. Methods to prepare and screen interfering RNA and select for the ability to block polynucleotide expression are known in the art and non-limiting examples of which are shown below. These interfering RNA are provided by this invention alone or in combination with a suitable vector or within a host cell. Compositions containing the RNAi are further provided. RNAi is useful to knock-out or knock-down select functions in a cell or tissue as known in the art and described infra. siRNA sequences can be designed by obtaining the target mRNA sequence and determining an appropriate siRNA complementary sequence. siRNAs of the invention are designed to interact with a target sequence, meaning they complement a target sequence sufficiently to hybridize to that sequence. An siRNA can be 100% identical to the target sequence. However, homology of the siRNA sequence to the target sequence can be less than 100% as long as the siRNA can hybridize to the target sequence. Thus, for example, the siRNA molecule can be at least 70%, 75%, 80%, 85%, 90%, 95%, 96%, 97%, 98%, 99% or 100% identical to the target sequence or the complement of the target sequence. Therefore, siRNA molecules with insertions, deletions or single point mutations relative to a target may also be used. The generation of several different siRNA sequences per target mRNA is recommended to allow screening for the optimal target sequence. A homology search, such as a BLAST search, should be performed to ensure that the siRNA sequence does not contain homology to any known mammalian gene.

In general, its preferable that the target sequence be located at least 100-200 nucleotides from the AUG initiation codon and at least 50-100 nucleotides away from the termination codon of the target mRNA (Duxbury (2004) J. Surgical Res. 117:339-344).

Researchers have determined that certain characteristics are common in siRNA molecules that effectively silence their target gene (Duxbury (2004) J. Surgical Res. 117:339-344; Ui-Tei et al. (2004) Nucl. Acids Res. 32:936-48). As a general guide, siRNAs that include one or more of the following conditions are particularly useful in gene silencing in mammalian cells: GC ratio of between 45-55%, no runs of more than 9 G/C residues, G/C at the 5′ end of the sense strand; A/U at the 5′ end of the antisense strand; and at least 5 A/U residues in the first 7 bases of the 5′ terminal of the antisense strand.

siRNA are, in general, from about 10 to about 30 nucleotides in length. For example, the siRNA can be 10-30 nucleotides long, 12-28 nucleotides long, 15-25 nucleotides long, 19-23 nucleotides long, or 21-23 nucleotides long. When an siRNA contains two strands of different lengths, the longer of the strands designates the length of the siRNA. In this situation, the unpaired nucleotides of the longer strand would form an overhang.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. For example, the stem can be 10-30 nucleotides long, 12-28 nucleotides long, 15-25 nucleotides long, 19-23 nucleotides long, or 21-23 nucleotides long. Tools to assist siRNA design are readily available to the public. For example, a computer-based siRNA design tool is available on the internet at www.dharmacon.com, last accessed on Nov. 26, 2007.

This invention also provides compositions for in vitro and in vivo use comprising, or alternatively consisting essentially of, or yet further consisting of one or more of the isolated polynucleotide as described herein and a pharmaceutically acceptable carrier. In one aspect, the compositions are pharmaceutical formulations for use in the therapeutic methods of this invention. In a further aspect, the invention provides a pharmaceutical composition comprising, or alternatively consisting essentially of, or yet further consisting of, the isolated polynucleotide in a concentration such that a therapeutically effective amount of the or pharmacological dose of the composition causes at least a 75%, or alternatively at least a 80%, or alternatively at least a 85%, or alternatively at least a 90%, or alternatively at least a 95% or alternatively at least a 97% reduction in viral infectivity when applied in a molar concentration of less than 1 micromolar, to a culture of responsive virus (e.g., influenza virus) virion, as compared to a control that does not receive the composition. In alternative aspects, the pharmacological dose of the composition when applied to the is in the range of about 150 nM to about 2 micromolar, or about 200 nM to about 2 micromolar, or about 250 nM to about 2 micromolar, or about 300 nM to about 2 micromolar, or about 400 nM to about 2 micromolar, or about 450 nm to about 2 micromolar, or about 500 nM to about 2 micromolar, or about 550 nM to about 2 micromolar, or about 600 nM to about 2 micromolar, or about 700 nM to about 2 micromolar, or about 800 nM to about 2 micromolar, or about 900 nM to about 2 micromolar, or about 1 micromolar to about 2 micromolar, or about 1.5 micromolar to about 2 micromolar, or about 50 nM to about 1 micromolar, or about 100 nM to about 1 micromolar, or about 150 nM to about 1 micromolar, or about 200 nM to about 1 micromolar, or about 250 nM to about 1 micromolar, or about 300 nM to about 1 micromolar, or about 400 nM to about 1 micromolar, or about 450 nm to about 1 micromolar, or about 500 nM to about 1 micromolar, or about 550 nM to about 1 micromolar, or about 600 nM to about 1 micromolar, or about 700 nM to about 1 micromolar, or about 800 nM to about 1 micromolar. Comparative effectiveness can be determined by suitable in vitro or in vivo methods as known in the art and described herein.

Synthesis of dsRNA and siRNA

dsRNA and siRNA can be synthesized chemically or enzymatically in vitro as described in Micura (2002) Agnes Chem. Int. Ed. Emgl. 41:2265-2269; Betz (2003) Promega Notes 85:15-18; and Paddison and Hannon (2002) Cancer Cell. 2:17-23. Chemical synthesis can be performed via manual or automated methods, both of which are well known in the art as described in Micura (2002), supra. siRNA can also be endogenously expressed inside the cells in the form of shRNAs as described in Yu et al. (2002) Proc. Natl. Acad. Sci. USA 99:6047-6052; and McManus et al. (2002) RNA 8:842-850. Endogenous expression has been achieved using plasmid-based expression systems using small nuclear RNA promoters, such as RNA polymerase III U6 or H1, or RNA polymerase II U1 as described in Brummelkamp et al. (2002) Science 296:550-553 (2002); and Novarino et al. (2004) J. Neurosci. 24:5322-5330.

In vitro enzymatic dsRNA and siRNA synthesis can be performed using an RNA polymerase mediated process to produce individual sense and antisense strands that are annealed in vitro prior to delivery into the cells of choice as describe in Fire et al. (1998) Nature 391:806-811; Donze and Picard (2002) Nucl. Acids Res. 30(10):e46; Yu et al. (2002); and Shim et al. (2002) J. Biol. Chem. 277:30413-30416. Several manufacturers (Promega, Ambion, New England Biolabs, and Stragene) produce transcription kits useful in performing the in vitro synthesis.

In vitro synthesis of siRNA can be achieved, for example, by using a pair of short, duplex oligonucleotides that contain T7 RNA polymerase promoters upstream of the sense and antisense RNA sequences as the DNA template. Each oligonucleotide of the duplex is a separate template for the synthesis of one strand of the siRNA. The separate short RNA strands that are synthesized are then annealed to form siRNA as described in Protocols and Applications, Chapter 2: RNA interference, Promega Corporation, (2005).

In vitro synthesis of dsRNA can be achieved, for example, by using a T7 RNA polymerase promoter at the 5′-ends of both DNA target sequence strands. This is accomplished by using separate DNA templates, each containing the target sequence in a different orientation relative to the T7 promoter, transcribed in two separate reactions. The resulting transcripts are mixed and annealed post-transcriptionally. DNA templates used in this reaction can be created by PCR or by using two linearized plasmid templates, each containing the T7 polymerase promoter at a different end of the target sequence. Protocols and Applications, Chapter 2: RNA interference, Promega Corporation, (2005).

RNA can be obtained by first inserting a DNA polynucleotide into a suitable prokaryotic or eukaryotic host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook and Russell (2001) supra. For instance, mRNA can be isolated using various lytic enzymes or chemical solutions according to the procedures set forth in Sambrook and Russell (2001) supra or extracted by nucleic-acid-binding resins following the accompanying instructions provided by manufactures.

In order to express the proteins described herein, delivery of nucleic acid sequences encoding the gene of interest can be delivered by several techniques. Examples of which include viral technologies (e.g. retroviral vectors, adenovirus vectors, adeno-associated virus vectors, alphavirus vectors and the like) and non-viral technologies (e.g. DNA/liposome complexes, micelles and targeted viral protein-DNA complexes) as described herein. Once inside the cell of interest, expression of the transgene can be under the control of ubiquitous promoters (e.g. EF-1) or tissue specific promoters (e.g. Calcium Calmodulin kinase 2 (CaMKI) promoter, NSE promoter and human Thy-1 promoter). Alternatively expression levels may controlled by use of an inducible promoter system (e.g. Tet on/off promoter) as described in Wiznerowicz et al. (2005) Stem Cells 77:8957-8961.

Non-limiting examples of promoters include, but are not limited to, the cytomegalovirus (CMV) promoter (Kaplitt et al. (1994) Nat. Genet. 8:148-154), CMV/human ÿ-globin promoter (Mandel et al. (1998) J. Neurosci. 18:4271-4284), NCX1 promoter, ÿMHC promoter, MLC2v promoter, GFAP promoter (Xu et al. (2001) Gene Ther., 8:1323-1332), the 1.8-kb neuron-specific enolase (NSE) promoter (Klein et al. (1998) Exp. Neurol. 150:183-194), chicken beta actin (CBA) promoter (Miyazaki (1989) Gene 79:269-277) and the β-glucuronidase (GUSB) promoter (Shipley et al. (1991) Genetics 10:1009-1018), the human serum albumin promoter, the alpha-1-antitrypsin promoter. To improve expression, other regulatory elements may additionally be operably linked to the transgene, such as, e.g., the Woodchuck Hepatitis Virus Post-Regulatory Element (WPRE) (Donello et al. (1998) J. Virol. 72: 5085-5092) or the bovine growth hormone (BGH) polyadenylation site.

The invention further provides the isolated polynucleotides of this invention operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells as described above and constructed using well known methods. See Sambrook and Russell (2001), supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; DEAE-dextran; electroporation; or microinjection. See Sambrook and Russell (2001), supra for this methodology.

The present invention also provides delivery vehicles suitable for delivery of a polynucleotide of the invention into cells (whether in vivo, ex vivo, or in vitro). A polynucleotide of the invention can be contained within a gene delivery vehicle, a cloning vector or an expression vector. These vectors (especially expression vectors) can in turn be manipulated to assume any of a number of forms which may, for example, facilitate delivery to and/or entry into a cell.

In one aspect when isolated polynucleotides encoding two or more peptides, at least one of which is SEQ ID NOS. 1 through 8 or 15 through 18 or 41 through 47, a biological equivalent or retro-inverso forms, are intended to be translated and optionally expressed, the polynucleotides encoding the peptides may be organized within a recombinant mRNA or cDNA molecule that results in the transcript that expresses on a single mRNA molecule the at least two peptides. This is accomplished by use of an polynucleotide that has the biological activity of an internal ribosome entry site (IRES) located between the polynucleotide encoding the two peptides. IRES elements initiate translation of polynucleotides without the use of a “cap” structure traditionally thought to be necessary for translation of proteins in eukaryotic cells. Initially described in connection with the untranslated regions of individual picornaviruses, e.g. polio virus and encephalomyocarditis virus, IRES elements were later shown to efficiently initiate translation of reading frames in eukaryotic cells and when positioned downstream from a eukaryotic promoter, it will not influence the “cap”-dependent translation of the first cistron. The IRES element typically is at least 450 nucleotides long when in occurs in viruses and possesses, at its 3′ end, a conserved “UUUC” sequence followed by a polypyrimidine trace, a G-poor spacer and an AUG triple.

As used herein, the term “IRES” is intended to include any molecule such as a mRNA polynucleotide or its reverse transcript (cDNA) which is able to initiate translation of the gene downstream from the polynucleotide without the benefit of a cap site in a eukaryotic cell. “IRES” elements can be identical to sequences found in nature, such as the picornavirus IRES, or they can be non-naturally or non-native sequences that perform the same function when transfected into a suitable host cell. Bi- and poly-cistronic expression vectors containing naturally occurring IRES elements are known in the art and described for example, in Pestova et al. (1998) Genes Dev. 12:67-83 and International Application No. WO 01/04306, which in turn on page 17, lines 35 to 38 references several literature references which include, but are not limited to Ramesh et al. (1996) Nucl. Acids Res. 24:2697-2700; Pelletier et al. (1988) Nature 334:320-325; January et al. (1989) J. Virol. 63:1651-1660; and Davies et al. (1992) J. Virol. 66:1924-1932. Paragraph [0009] of U.S. Patent Appl. Publ. No.: 2005/0014150 A1 discloses several issued U.S. patents wherein a virally-derived IRES element was used to express foreign gene(s) in linear multi-cistronic mRNAs in mammalian cells, plant cells and generally in eukaryotic cells. U.S. Patent Appl. Publ. No. 2004/0082034 A1 discloses an IRES element active in insect cells. Methods to identify new elements also are described in U.S. Pat. No. 6,833,254.

Also within the term “IRES” element are cellular sequences similar to that disclosed in U.S. Pat. No. 6,653,132. The patent discloses a sequence element (designated SP163) composed of sequences derived from the 5′-UTR of VEGF (Vascular Endothelial Growth Factor gene), which, was presumably generated through a previously unknown mode of alternative splicing. The patentees report that an advantages of SP163 is that it is a natural cellular IRES element with a superior performance as a translation stimulator and as a mediator of cap-independent translation relative to known cellular IRES elements and that these functions are maintained under stress conditions.

Further within the term “IRES” element are artificial sequences that function as IRES elements that are described, for example, in U.S. Patent Appl. Publ. No.: 2005/0059004 A1.

Operatively linked to the IRES element and separately, are sequences necessary for the translation and proper processing of the peptides. Examples of such include, but are not limited to a eukaryotic promoter, an enhancer, a termination sequence and a polyadenylation sequence. Construction and use of such sequences are known in the art and are combined with IRES elements and protein sequences using recombinant methods. “Operatively linked” shall mean the juxtaposition of two or more components in a manner that allows them to junction for their intended purpose. Promoters are sequences which drive transcription of the marker or target protein. It must be selected for use in the particular host cell, i.e., mammalian, insect or plant. Viral or mammalian promoters will function in mammalian cells. The promoters can be constitutive or inducible, examples of which are known and described in the art.

In one aspect, the peptides are transcribed and translated from a separate recombinant polynucleotide and combined into a functional protein in the host cell. This recombinant polynucleotide does not require the IRES element or marker protein although in one aspect, it may be present.

These isolated host cells containing the polynucleotides of this invention are useful in the methods described herein as well as for the recombinant replication of the polynucleotides and for the recombinant production of peptides and for high throughput screening.

### Host Cells

Also provided are isolated host cells comprising one or more of the polypeptides, peptide fragments and/or polynucleotides of this invention. Suitable cells containing the inventive polypeptides and/or polynucleotides include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escerichia coli, Salmonella enterica and Streptococcus gordonii. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

In addition to species specificity, the cells can be of any particular tissue type such as neuronal or alternatively a somatic or embryonic stem cell such as a stem cell that can or can not differentiate into a neuronal cell, e.g., embryonic stem cell, an induced pluripotent embryonic stem cell (iPSC), adipose stem cell, neuronal stem cell and hematopoietic stem cell. The stem cell can be of human or animal origin, such as mammalian.

### Therapeutic Antibody Compositions

This invention also provides an antibody capable of specifically forming a complex with a protein or peptide or peptide fragment of this invention, which are useful in the therapeutic methods of this invention, e.g. the proteins and peptide fragments identified in Tables 1 through 4 and 6, supra. The term “antibody” includes polyclonal antibodies and monoclonal antibodies, antibody fragments, as well as derivatives thereof (described above). The antibodies include, but are not limited to mouse, rat, and rabbit or human antibodies. limited to cows, rabbits, goats, mice, rats, hamsters, guinea pigs, sheep, dogs, cats, monkeys, chimpanzees, apes, etc. The antibodies are also useful to identify and purify therapeutic and/or diagnostic polypeptides. Also provided are hybridoma cell lines producing monoclonal antibodies of this invention.

Polyclonal antibodies of the invention can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammal's serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment, hybrid or biological equivalent of each thereof, of the present invention. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) Bioinvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al., (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al., Proc. Natl. Acad. Sci. USA (1996) 93:7843-7848); gel microdroplet and flow cytometry (Powell et al. (1990) Biotechnol. 8:333-337; One Cell Systems, (Cambridge, Mass.).; Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134).

Antibody derivatives of the present invention can also be prepared by delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured there from. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to known methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See, e.g., Russel et al. (2000) Infection and Immunity April 2000:1820-1826; Gallo et al. (2000) European J. of Immun. 30:534-540; Green (1999) J. of Immun. Methods 231:11-23; Yang et al. (1999) J. of Leukocyte Biology 66:401-410; Yang (1999) Cancer Research 59(6):1236-1243; Jakobovits (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H}1-VH-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

If a monoclonal antibody being tested binds with protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. (1985) Proc. Natl. Acad. Sci. USA 82:8653 or Spira, et al. (1984) J. Immunol. Methods 74:307.

The isolation of other hybridomas secreting monoclonal antibodies with the specificity of the monoclonal antibodies of the invention can also be accomplished by one of ordinary skill in the art by producing anti-idiotypic antibodies. Herlyn, et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

The invention also provides antibodies that not only bind to a peptide fragment as identified herein but are further characterized by blocking vFLIP or cFLIP binding to Atg3. The blocking antibodies are identified using methods well know in the art.

Antibodies can be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), (bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

### Compositions for Therapy

One or more of the above host cell, antibody, antibody fragment, antibody derivative, protein, hybrid peptide, variant peptide, peptide fragment or polynucleotide encoding these compositions can be further combined with a carrier, a pharmaceutically acceptable carrier or medical device which is suitable for use of the compositions in diagnostic or therapeutic methods. Thus, the compositions comprise, or alternatively consist essentially of, or yet further consists of, one or more of the above compositions described above in combination with a carrier, a pharmaceutically acceptable carrier or medical device.

The carrier can be a liquid phase carrier or a solid phase carrier, e.g., bead, gel, microarray, or carrier molecule such as a liposome. The composition can optionally further comprise at least one further compound, protein or composition.

Additional examples of “carriers” includes therapeutically active agents such as another peptide or protein (e.g., an Fab′ fragment). For example, an antibody of this invention, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this invention encompasses a large variety of antibody conjugates, bi- and multispecific molecules, and fusion proteins, whether or not they target the same epitope as the antibodies of this invention.

Yet additional examples of carriers are organic molecules (also termed modifying agents) or activating agents, that can be covalently attached, directly or indirectly, to an antibody of this invention. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the invention has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-Δ9-octadecanoate, all cis-Δ5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The present invention provides a composition comprising, or alternatively consisting essentially of, or yet further consisting of, at least one antibody of this invention, derivative or fragment thereof, suitable for administration in an effective amount to increase or induce cell cancer death, eliminate viral particles associated with a viral infection, and/or treat or ameliorate a neurodegenerative disease. The compositions include, for example, pharmaceutical and diagnostic compositions/kits, comprising a pharmaceutically acceptable carrier and at least one antibody of this invention, variant, derivative or fragment thereof. As noted above, the composition can further comprise additional antibodies or therapeutic agents which in combination, provide multiple therapies tailored to provide the maximum therapeutic benefit.

Alternatively, a composition of this invention can be co-administered with other therapeutic agents, whether or not linked to them or administered in the same dosing. They can be co-administered simultaneously with such agents (e.g., in a single composition or separately) or can be administered before or after administration of such agents. Such agents can include Aricept® (donepezil), Razadyne® (galantamine), Nanenda® (mementine), Exalon® (rivastigmine), Cognex® (tacrine), or other agents known to those skilled in the art. The compositions can be combined with alternative therapies such as administration of tranquilizers, mood stabilizing medications, behavior treatments (including treatments for aggressive behavior, incontinence, sleep difficulties, and wandering behavior), and individual activities and therapies (e.g., Reminiscence therapy) known to those skilled in the art.

### Compositions for Diagnosis and Therapy

One or more of the above compositions can be further combined with a carrier, a pharmaceutically acceptable carrier or medical device which is suitable for use of the compositions in diagnostic or therapeutic methods.

The carrier can be a liquid phase carrier or a solid phase carrier, e.g., bead, gel, gene chip, microarray, or carrier molecule such as a liposome. The composition can optionally further comprise at least one further compound, protein or composition.

Additional examples of “carriers” includes therapeutically active agents such as another peptide or protein (e.g., an Fab′ fragment). For example, an antibody of this invention, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this invention encompasses a large variety of antibody conjugates, bi- and multispecific molecules, and fusion proteins, whether or not they target the same epitope as the antibodies of this invention.

Yet additional examples of carriers are organic molecules (also termed modifying agents) or activating agents, that can be covalently attached, directly or indirectly, to an antibody of this invention. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the invention has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-Δ9-octadecanoate, all cis-Δ5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

Also provided is a composition containing at least one antibody of this invention, derivative or fragment thereof, suitable for administration in an effective amount to modulate a neurodegenerative disorder correlative to the expression of the receptor or receptor complex. The compositions include, for example, pharmaceutical and diagnostic compositions/kits, comprising a pharmaceutically acceptable carrier and at least one antibody of this invention, variant, derivative or fragment thereof. As noted above, the composition can further comprise additional antibodies or therapeutic agents which in combination, provide multiple therapies tailored to provide the maximum therapeutic benefit.

Alternatively, a composition of this invention can be co-administered with other therapeutic agents, whether or not linked to them or administered in the same dosing. They can be co-administered simultaneously with such agents (e.g., in a single composition or separately) or can be administered before or after administration of such agents. Such agents can include anticancer therapies such as irinotecan, 5-Fluorouracil, Erbitux, Cetuximab, FOLFOX, radiation therapy, or therapies for neurodegenerative disorders such as Aricept® (donepezil), Razadyne® (galantamine), Nanenda® (mementine), Exalon® (rivastigmine), Cognex® (tacrine), or other agents known to those skilled in the art. The compositions can be combined with alternative therapies such as administration of tranquilizers, mood stabilizing medications, behavior treatments (including treatments for aggressive behavior, incontinence, sleep difficulties, and wandering behavior), and individual activities and therapies (e.g., Reminiscence therapy) known to those skilled in the art. Also provided is a kit comprising any of the compositions of the invention and instructions for use it for any method of the invention.

### Diagnostic Methods Utilizing Recombinant DNA Technology and Bioinformatics

The polynucleotides of this invention can be attached to a solid support such as an array or high density chip for use in high throughput screening assays using methods known in the art. For example, the polynucleotide of SEQ ID NOS. 1 through 8 or 41 through 47 can be used as a probe to identify expression in a subject sample. International PCT Application No. WO 97/10365 and U.S. Pat. Nos. 5,405,783; 5,412,087 and 5,445,934, for example, disclose the construction of high density oligonucleotide chips which can contain one or more sequences. The chips can be synthesized on a derivatized glass surface using the methods disclosed in U.S. Pat. Nos. 5,405,783; 5,412,087 and 5,445,934. Photoprotected nucleoside phosphoramidites can be coupled to the glass surface, selectively deprotected by photolysis through a photolithographic mask, and reacted with a second protected nucleoside phosphoramidite. The coupling/deprotection process is repeated until the desired probe is complete.

One can use chemical synthesis to provide the isolated polynucleotides of the present invention. Chemical synthesis of polynucleotides can be accomplished using a number of protocols, including the use of solid support chemistry, where an oligonucleotide is synthesized one nucleoside at a time while anchored to an inorganic polymer. The first nucleotide is attached to an inorganic polymer using a reactive group on the polymer which reacts with a reactive group on the nucleoside to form a covalent linkage. Each subsequent nucleoside is then added to the first nucleoside molecule by: 1) formation of a phosphite linkage between the original nucleoside and a new nucleoside with a protecting group; 2) conversion of the phosphite linkage to a phosphate linkage by oxidation; and 3) removal of one of the protecting groups to form a new reactive site for the next nucleoside as described in U.S. Pat. Nos. 4,458,066; 5,153,319; 5,132,418; 4,973,679 all of which are incorporated by reference herein. Solid phase synthesis of oligonucleotides eliminates the need to isolate and purify the intermediate products after the addition of every nucleotide base. Following the synthesis of RNA, the oligonucleotides is deprotected (U.S. Pat. No. 5,831,071) and purified to remove by-products, incomplete synthesis products, and the like.

U.S. Pat. No. 5,686,599, describes a method for one pot deprotection of RNA under conditions suitable for the removal of the protecting group from the 2′ hydroxyl position. U.S. Pat. No. 5,804,683, describes a method for the removal of exocyclic protecting groups using alkylamines. U.S. Pat. No. 5,831,071, describes a method for the deprotection of RNA using ethylamine, propylamine, or butylamine. U.S. Pat. No. 5,281,701, describes methods and reagents for the synthesis of RNA using 5′-O-protected-2′-O-alkylsilyl-adenosine phosphoramidite and 5′-O-protected-2′-O-alkylsilylguanosine phosphoramidite monomers which are deprotected using ethylthiotetrazole. Usman and Cedergren (1992) Trends in Biochem. Sci. 17:334-339 describe the synthesis of RNA-DNA chimeras for use in studies of the role of 2′ hydroxyl groups. Sproat et al. (1995) Nucleosides & Nucleotides 14:255-273, describe the use of 5-ethylthio-1H-tetrazole as an activator to enhance the quality of oligonucleotide synthesis and product yield. Gait et al. (1991) Oligonucleotides and Analogues, ed. F. Eckstein, Oxford University Press 25-48, describe general methods for the synthesis of RNA. U.S. Pat. Nos. 4,923,901; 5,723,599; 5,674,856; 5,141,813; 5,419,966; 4,458,066; 5,252,723; Weetall et al. (1974) Methods in Enzymology 34:59-72; Van Aerschot et al. (1988) Nucleosides and Nucleotides 7:75-90; Maskos and Southern (1992) Nucleic Acids Research 20: 1679-1684; Van Ness et al. (1991) Nucleic Acids Research 19:3345-3350; Katzhendler et al. (1989) Tetrahedron 45:2777-2792; Hovinen et al. (1994) Tetrahedron 50:7203-7218; GB 2,169,605; EP 325,970; PCT publication No. WO 94/01446; German Patent No. 280,968; and BaGerman Patent No. 4,306,839, all describe specific examples of solid supports for oligonucleotide synthesis and specific methods of use for certain oligonucleotides. Additionally, methods and reagents for oligonucleotide synthesis as know to one of skill in the are as describe by U.S. Pat. No. 7,205,399, incorporated herein by reference in its entirety.

One can use these compositions to monitor or detect the expression level of a gene of interest such as Atg3 or FLIP through exposure of a sample suspected of containing the polynucleotide to an chip containing a probe that specifically recognizes and binds the gene of interest. Extracted nucleic acid is labeled, for example, with a detectable label, preferably during an amplification step. Hybridization of the labeled sample is performed at an appropriate stringency level. The degree of probe-nucleic acid hybridization is quantitatively measured using a detection device, such as a confocal microscope. See, U.S. Pat. Nos. 5,578,832 and 5,631,734. The obtained measurement is directly correlated with gene expression level.

The probes and high density oligonucleotide probe arrays also provide an effective means of monitoring expression of a multiplicity of genes, one of which includes the gene. Thus, the expression monitoring methods can be used in a wide variety of circumstances including detection of disease, identification of differential gene expression between samples isolated from the same patient over a time course, or screening for compositions that upregulate or downregulate the expression of the gene at one time, or alternatively, over a period of time.

Detectable labels suitable for use in the present invention include those identified above as well as any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P) enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

Patent Publication WO 97/10365 describes methods for adding the label to the target (sample) nucleic acid(s) prior to or alternatively, after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids, see Laboratory Techniques In Biochemistry And Molecular Biology, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using the methods disclosed in International PCT Application No. WO 97/10365.

Results from the chip assay are typically analyzed using a computer software program. See, for example, EP 0717 113 A2 and WO 95/20681. The hybridization data is read into the program, which calculates the expression level of the targeted gene(s). The figure is compared against existing data sets of gene expression levels for diseased and healthy individuals. A correlation between the obtained data and that of a set of diseased individuals indicates the onset of a disease in the subject patient.

### Diagnostic Methods for Detecting and Quantifying Protein or Polypeptides

This invention also provided methods for detecting the pro-autophagy complex expression using the compositions described above. A variety of techniques are available in the art for protein analysis and include, but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays and PAGE-SDS, mass spectrometry. As is apparent to those skilled in the art, a positive and a negative control can by assayed concurrently to verify the integrity of the results.

### Methods to Identify Therapeutic Agents

The present invention also provides methods to identify leads and methods for cancer and/or neurodegenerative disorders. In one aspect, the screen identifies lead compounds or biological agents that mimic the peptides or fragments identified above and which are useful to treat these disorders or to treat or ameliorate the symptoms associated with the disorders. Test substances for screening can come from any source. They can be libraries of natural products, combinatorial chemical libraries, biological products made by recombinant libraries, etc. The source of the test substances is not critical to the invention. The present invention provides means for screening compounds and compositions which may previously have been overlooked in other screening schemes.

To practice the screen or assay in vitro, suitable cell cultures or tissue cultures are first provided. The cell can be a cultured cell or a genetically modified cell which differentially expresses the receptor and/or receptor complex. Alternatively, the cells can be from a tissue culture as described below. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, suitable cells may be cultured in microtiter plates and several agents may be assayed at the same time by noting genotypic changes, phenotypic changes and/or cell death.

When the agent is a composition other than a DNA or RNA nucleic acid molecule, the suitable conditions may be by directly added to the cell culture or added to culture medium for addition. As is apparent to those skilled in the art, an “effective” amount must be added which can be empirically determined.

The screen involves contacting the agent with a test cell expressing the complex and then assaying the cell its ability to provide a biological response similar to cFLIP, vFLIP or fragments identified above, or alternatively for binding of the agent to the Atg3 complex. In yet another aspect, the test cell or tissue sample is isolated from the subject to be treated and one or more potential agents are screened to determine the optimal therapeutic and/or course of treatment for that individual patient.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies. They can be administered concurrently or sequentially.

Use of the screen in an animal such as a rat or mouse, the method provides a convenient animal model system which can be used prior to clinical testing of the therapeutic agent or alternatively, for lead optimization. In this system, a candidate agent is a potential drug, and may therefore be suitable for further development, if the agent binds the receptor or receptor complex each as compared to untreated, animal expressing the receptor and/or complex. It also can be useful to have a separate negative control group of cells or animals which are healthy and not treated, which provides a further basis for comparison.

### Methods of Use of FLIP Proteins and Their Compositions

Applicants have discovered that vFLIP and cFLIP proteins are useful for competing with LC3 for Atg3 binding in the LC3-Atg4-Atg7-Atg3 conjugation complex which is necessary for the induction of autophagy, resulting in the diminishing or suppressing of autophagic cell death. Thus, in another aspect this invention provides a method of diminishing or inhibiting the formation of the LC3-Atg4-Atg7-Atg3 conjugation complex that is necessary for the induction of autophagy, by administering an effective amount of vFLIP or cFLIP protein that competes against LC3 for the binding of Atg3.

In one aspect, the vFLIP or cFLIP protein that is administered in this method is a full-length protein comprising, or alternatively consisting essentially of, or yet further consisting of an amino acid sequence listed in Table 2 (SEQ ID NO. 9, SEQ ID NO. 10, SEQ ID NO. 11, or SEQ ID NO. 12). In some embodiments, the vFLIP or cFLIP protein that is administered is a polypeptide that is substantially homologous and biologically equivalent to SEQ ID NO. 9, SEQ ID NO. 10, SEQ ID NO. 11, or SEQ ID NO. 12. Substantially homologous and biologically equivalent polypeptides intend those having at least 80% homology, or alternatively at least 85% homology, or alternatively at least 90% homology, or alternatively, at least 95% homology or alternatively, at least 98% homology to SEQ ID NO. 9, SEQ ID NO. 10, SEQ ID NO. 11, or SEQ ID NO. 12, each as determined using methods known to those skilled in the art and identified herein, when run under default parameters. Preferred amino acid substitutions for the biologically equivalent peptides are described supra. Also within the scope of this invention are the retro-inverso forms of these peptides.

In another aspect, the cFLIP protein that is administered in this method is a short form of the cFLIP protein. In some embodiments, the cFLIP protein that is administered is a polypeptide that is substantially homologous and biologically equivalent to SEQ ID NO. 13. Substantially homologous and biologically equivalent polypeptides intend those having at least 80% homology, or alternatively at least 85% homology, or alternatively at least 90% homology, or alternatively, at least 95% homology or alternatively, at least 98% homology to SEQ ID NO. 13, each as determined using methods known to those skilled in the art and identified herein, when run under default parameters. Preferred amino acid substitutions for the biologically equivalent peptides are described supra. Also within the scope of this invention are the retro-inverso forms of these peptides.

A protein or polypeptide of this method can be administered into cells (whether in vivo, ex vivo, or in vitro) using any delivery vehicle suitable for delivery of a polypeptide. The polypeptide can be directly administered, or the polypeptide can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. Polypeptides of this invention also can be combined with various liquid phase carriers, such as sterile or aqueous solutions, pharmaceutically acceptable carriers, suspensions and emulsions.

Lack of inhibition of autophagy and autophagic is implicated in some tumor cell growth. The administration of a vFLIP or a cFLIP protein or equivalent thereof, or alternatively a polynucleotide encoding a the vFLIP or cFLIP protein or equivalent thereof is useful for competing with LC3 for Atg3 binding in the LC3-Atg4-Atg7-Atg3 conjugation complex which is necessary for the induction of autophagy, resulting in the diminishing or suppressing of autophagic cell death in a precancerous cell or a cancer cell that has been previously or subsequently exposed to chemotherapy. Thus, in yet another aspect, this invention provides methods of inhibiting or diminishing autophagy by administering to the subject in need thereof an effective amount of a vFLIP or cFLIP protein or a polynucleotide that encodes such, and as described herein. Administration can be by any suitable method and effective amounts can be empirically determined by a treating physician. The proteins or polynucleotides can be delivered alone or in combination with another active agent. In one aspect, the cancer cell is a leukemia cell, a lymphoma cell or a solid tumor cell. In another aspect, the cancer cell is a leukemia cell, a lymphoma cell, a lung tumor cell, a non-small cell lung tumor cell, a breast tumor cell, a head and neck tumor cell, an ovarian tumor cell, a colon tumor cell, a rectal tumor cell, a colorectal tumor cell, an esophageal tumor cell, a gastric tumor cell, a liver tumor cell, a bone tumor cell, a spleen tumor cell, a pancreatic tumor cell, or a gallbladder tumor cell. In yet another aspect, the tumor cell is a leukemia cell, a prostate tumor cell or a colon tumor cell.

It is discovered herein that the compositions of the invention induced cell deaths of cancer cells of various origins. Accordingly, in another aspect, the invention provides methods of suppressing growth of a cancer or tumor cell, or increasing or inducing death of tumor cell, by administering to the subject a therapeutically effective amount of a FLICE-like inhibitor protein, thereby suppressing growth of the cancer or tumor cell. Polypeptides and polynucleotides and other agents that induce autophagy are described above. In one aspect, the cancer cell is a leukemia cell, a lymphoma cell or a solid tumor cell. In another aspect, the cancer cell is a leukemia cell, a lymphoma cell, a lung tumor cell, a non-small cell lung tumor cell, a breast tumor cell, a head and neck tumor cell, an ovarian tumor cell, a colon tumor cell, a rectal tumor cell, a colorectal tumor cell, an esophageal tumor cell, a gastric tumor cell, a liver tumor cell, a bone tumor cell, a spleen tumor cell, a pancreatic tumor cell, or a gallbladder tumor cell. In yet another aspect, the tumor cell is a leukemia cell, a prostate tumor cell or a colon tumor cell.

In some embodiments, the polypeptide described herein that may be administered therapeutically by this method may comprise one or more of the full length vFLIP or cFLIP proteins. In other embodiments, the polypeptide described herein that may be administered therapeutically by this method may comprise a short form of cFLIP. In some embodiments, the polypeptide described herein that may be administered by this method may comprise one or more of the amino acid sequences listed in Table 2. In some embodiments, the polypeptide described herein that may be administered therapeutically by this method may comprise a substantially homologous and biologically equivalent polypeptide having at least 80% homology, or alternatively at least 85% homology, or alternatively at least 90% homology, or alternatively, at least 95% homology or alternatively, at least 98% homology to SEQ ID NO. 9, SEQ ID NO. 10, SEQ ID NO. 11, SEQ ID NO. 12, or SEQ ID NO. 13, or variant or hybrid peptides between any two of them or their biological equivalents, each as determined using methods known to those skilled in the art and identified herein, when run under default parameters. Preferred amino acid substitutions for the biologically equivalent peptides are described supra. Also within the scope of this invention are the retro-inverso forms of these peptides. In alternate embodiments, the polynucleotide encoding the polypeptide is administered or delivered to the cell or a subject in need thereof.

In therapeutic applications, a pharmaceutical composition containing one or more polypeptide or polypeptide described herein is administered to a patient suspected of, or already suffering from such a disease associated with the regulation of autophagy, wherein said composition is administered in an amount sufficient to cure, or at least partially arrest, the symptoms of the disease (biochemical, histological and/or behavioral), including its complication and intermediate pathological phenotypes in development of the disease.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the polypeptide of this invention to decrease autophagy either in vitro or in vivo by at least 10%, 25%, 40%, 60%, 80%, 90% or 95% as compared to control. Determination of these parameters is well within the skill of the art. These considerations, as well as effective formulations and administration procedures are well known in the art and are described in standard textbooks.

The “therapeutically effective amount” will vary depending on the polypeptide, the disease and its severity and the age, weight, etc., of the patient to be treated all of which is within the skill of the attending clinician. It is contemplated that a therapeutically effective amount of a polypeptide described herein will decrease levels of autophagy in the patient as compared to the levels of autophagy in the absence of treatment. As such, cancer growth is suppressed or decreased. A therapeutically effective amount is distinguishable from an amount having a biological effect (a “biologically effective amount”). A polypeptide of the present invention may have one or more biological effects in vitro or even in vivo, such as decreasing binding of LC3 to Atg3 or decreasing the level of autophagy in a cell. A biological effect, however, may not result in any clinically measurable therapeutically effect as described above as determined by methods within the skill of the attending clinician.

### Methods of Use of Isolated Peptide Fragments and Their Compositions

Certain isolated peptides and peptide fragments of this invention are useful for competing with full-length FLIP protein for Atg3 binding in a dosage-dependent manner, resulting in an increase in autophagic cell death. Thus, in another aspect this invention provides a method of diminishing or inhibiting the binding of vFLIP or cFLIP to Atg3 that is necessary for diminishing or inhibiting autophagy, by administering isolated peptide fragments of vFLIP or cFLIP, or compositions comprising isolated peptide fragments of vFLIP or cFLIP or hybrid or variant peptides or biological equivalents of each thereof, that are capable of competing against vFLIP or cFLIP for the binding of Atg3. These can be combined with a transduction domain or and/or administered in the retro-inverso form, as well as biological equivalents of each thereof. In an alternate embodiment, a polynucleotide encoding the isolated peptide encoding the above noted peptides identified herein is delivered to the cell or administered to the subject.

Yet another aspect this invention provides a method of increasing or inducing death of a precancerous cell, by administering to the cell an effective amount of a FLIP peptide fragment isolated from a FLICE-like inhibitor protein (FLIP protein), wherein the peptide fragment is capable of competing against a full-length FLIP protein for binding of an Atg3 protein in a LC3-Atg4-Atg7-Atg3 conjugation complex, thereby increasing or inducing autophagy and increasing or inducing death of the precancerous cell. In one aspect, an effective amount of a chemotherapeutic agent is co-adminitered, concurrently, subsequently or prior to administration of the FLIP protein, polynucleotide, variant, hybrid or equivalent thereof.

An isolated peptide fragment, variant, hybrid, polynucleotide, polypeptide, antibody, or composition of this method can be administered into cells (whether in vivo, ex vivo, or in vitro) using any delivery vehicle suitable for delivery of a peptide fragment, polynucleotide, polypeptide, antibody, or composition. An isolated protein, peptide fragment or polypeptide can be directly administered, or a protein, peptide fragment, variant, hybrid or polynucleotide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. Peptide fragments or polypeptides of this invention also can be combined with various liquid phase carriers, such as sterile or aqueous solutions, pharmaceutically acceptable carriers, suspensions and emulsions. A polynucleotide of the invention can be contained within a gene delivery vehicle, a cloning vector or an expression vector. These vectors (especially expression vectors) can in turn be manipulated to assume any of a number of forms which may, for example, facilitate delivery to and/or entry into a cell. An antibody of this invention can be functionally linked (e.g., by chemical coupling, genetic fusion, noncovalent association or otherwise) to one or more other molecular entities for administration by this method. For example, an antibody of this invention can be linked to another antibody (e.g., to produce a bispecific or a multispecific antibody), a cytotoxin, a cellular ligand or an antigen. A composition can be further combined with a carrier, a pharmaceutically acceptable carrier or medical device for administration by this method.

In one aspect, the isolated peptide fragment, variant, hybrid, polynucleotide, polypeptide, antibody, or composition described herein that may be administered therapeutically by this method may comprise one or more of the amino acid sequences corresponding to an alpha helix region of a death effector domain of vFLIP or cFLIP. In some embodiments, the isolated peptide fragments, polynucleotide, polypeptide, antibody, or composition described herein that may be administered by this method may comprise, or alternatively consisting essentially of, or yet further consisting of, one or more of the amino acid sequences listed in Table 1 (SEQ ID NOS: 1 through 8 or 15 through 18 or 41 through 47, listed in Table 6 and variations thereof as described above). In some embodiments, the isolated peptide fragments, polynucleotide, polypeptide, antibody, or composition described herein that may be administered therapeutically by this method may comprise comprise, or alternatively consisting essentially of, or yet further consisting of, a “biologically equivalent” or “biologically active” peptide fragment encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. For example, one or more of the valine, isoleucine, leucine, methionine, phenylalanine, or tryptophan residues of the hydrophobic core of an alpha helix of a death effector domain may be modified or substituted with another hydrophobic residue such as valine, isoleucine, leucine, methionine, phenylalanine, or tryptophan.

In therapeutic applications, a pharmaceutical composition containing one or more host cell, protein, peptide fragment, variant, hybrid, polynucleotide, polypeptide, antibody, or composition described herein is administered to a patient suspected of, or already suffering from such a disease associated with the regulation of autophagy, wherein said composition is administered in an amount sufficient to cure, or at least partially arrest, the symptoms of the disease (biochemical, histological and/or behavioral), including its complication and intermediate pathological phenotypes in development of the disease.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the peptide fragment, polypeptide, polynucleotide, antibody, or compositions of this invention to increase autophagy either in vitro or in vivo by at least 10%, 25%, 40%, 60%, 80%, 90% or 95% as compared to control. Determination of these parameters is well within the skill of the art. These considerations, as well as effective formulations and administration procedures are well known in the art and are described in standard textbooks.

The “therapeutically effective amount” will vary depending on the peptide fragment, polypeptide, polynucleotide, or compositions, the disease and its severity and the age, weight, etc., of the patient to be treated all of which is within the skill of the attending clinician. It is contemplated that a therapeutically effective amount of one or more of a peptide fragment, polynucleotide, polypeptide, antibody or composition described herein will increase levels of autophagy in the patient as compared to the levels of autophagy in the absence of treatment. As such, cancer cell death is increased, the number of viral particles eliminated from the patient is increased, or the symptoms or effects of neurodegenerative disease are ameliorated. A therapeutically effective amount is distinguishable from an amount having a biological effect (a “biologically effective amount”). A peptide fragment, polypeptide, polynucleotide, or compositions of the present invention may have one or more biological effects in vitro or even in vivo, such as decreasing binding of FLIP protein to Atg3 or increasing the level of autophagy in a cell. A biological effect, however, may not result in any clinically measurable therapeutically effect as described above as determined by methods within the skill of the attending clinician.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active agent at sites of disease. This may be achieved, for example, by the intravenous injection of the agent, optionally in saline, or orally administered, for example, as a tablet, capsule or syrup containing the active ingredient. Desirable blood levels of the agent may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of the agent through the skin or other affected areas. Examples of such dermal penetration enhancers include dimethylsulfoxide and related analogues.

The oily phase of the emulsions of this invention may be constituted from known ingredients in an known manner. While this phase may comprise merely an emulsifier (otherwise known as an emulgent), it desirably comprises a mixture of at lease one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

### Kits and Screens

Also disclosed herein are kits having components and instructions for inducing autophagy in a cell or in a subject in need thereof, or for inducing autophagy in a cancer cell, or inhibiting viral entry into a cell. The kits contain instructions for use and one or more of a FLIP peptide fragment, variant, hybrid or biological equivalent thereof, an amino acid comprising SEQ ID. NOS. 1 through 8, 15 through 18, 41 through 47 or a biological equivalent of each thereof, a FLIP peptide fragment isolated from a FLIP protein death effector domain region of the FLIP protein or from a portion of the death effector domain region of the FLIP protein; and a FLIP peptide fragment isolated from an alpha-helix fragment of the FLIP protein death effector domain region or from a portion of the alpha-helix fragment of the FLIP protein death effector domain region, or a peptide, polynucleotide, antibody, host cell or composition as described above.

Yet further disclosed is a screen to identify possible therapeutic agents comprising contacting a first cell or tissue sample with a test agent and a second cell or tissue agent that is disclosed herein to induce autophagy or alternatively, inhibit it. Examples of such disclosed agents include without limitation. one or more of a FLIP protein, a FLIP peptide fragment, variant, hybrid or biological equivalent thereof, an amino acid comprising SEQ ID. NOS. 1 through 8, 15 through 18, 41 through 47 or a biological equivalent of each thereof, a FLIP peptide fragment isolated from a FLIP protein death effector domain region of the FLIP protein or from a portion of the death effector domain region of the FLIP protein; and a FLIP peptide fragment isolated from an alpha-helix fragment of the FLIP protein death effector domain region or from a portion of the alpha-helix fragment of the FLIP protein death effector domain region. The contacting of the first and second samples is performed under conditions that promote or inhibit autophagy of the cell or in the tissue (as necessary for the test agent's presumed activity), and comparing the autophagy of the first sample with the second sample, wherein the test agent is a possible therapeutic agent if the autophagy of the first sample is the same or similar to the second sample, thereby screening for possible therapeutic agents. Agents for the uses as described herein such as to induce autophagy in a cancer cell can be screened by high throughput screening.

The following examples are intended to illustrate, and not limit, the inventions disclosed herein.

### Experimental Examples

### Example 1

### FLIP Suppresses Autophagy

To identify additional anti-autophagic viral proteins, a KSHV expression library was screened by evaluating a GFP-LC3 staining pattern. Autophagic stimulation was induced, and candidate proteins were identified on the basis of exhibiting a redistribution of GFP-LC3 staining, from a diffused staining pattern throughout the cytoplasm and nucleus to a cytoplasmic punctate structure specifically labeling preautophagosomal and autophagosomal membranes. (Mizushima et al. (2004) Mol. Cell. Biol. 15:1101-1111). The screen identified that vFLIP (also called K13) effectively suppressed starvation- and rapamycin-induced autophagy, evidenced by the reduction of GFP-LC3 puncta.

At 12-16 hours post-transfection with GFP-LC3, NIH3T3 cells containing vector or the FLIP gene were treated with Hank's solution for 4 hr or with 2 μM rapamycin for 3 hr. Subsequently, GFP-LC3 was detected using an inverted fluorescence microscope, autophagy was quantified as means (±SD) of the combined results from three independent experiments, and autophagosomes were visualized through scanning electron microscopy. NIH3T3-Vector, NIH3T3-KSHV-vFLIP, and NIH3T3-MCV-159L cells were treated with Hank's solution for 4 hr, followed by immunoblotting with anti-LC3 and anti-βTubulin. At 12-16 hr post-transfection with GFP-LC3, TREX-BCBL-Vector and TREX-BCBL-vFLIP cells were treated with doxycycline for 24 hr, followed by incubation with 2 μM rapamycin for an additional 12 hr. The detection of GFP-LC3 and autophagy levels was performed as described above, vFLIP expression was detected by IB with anti-Flag, and autophagosomes were visualized by scanning EM. TNF-induced apoptosis and NF-κB luciferase assay were performed as previously described (Matta and Chaudhary (2004) Proc. Natl. Acad. Sci. USA 101:9399-9404).

The anti-autophagic properties of vFLIP, as well as the related cFLIP, HSV, and MCV, were evaluated by ectopic expression of HVS, vFLIP, MCV 159L, and the short form of cFLIP (cFLIPₛ) in NIHI3T3 cells. DNA fragments corresponding to the coding sequences of the KSHV-vFLIP, human cFLIPₛ, MCV-MC159, and HVS-vFLIP genes were amplified via polymerase chain reaction and subcloned into pcDNA5/FRT/TO between the AflII and BamHI restriction sites or pEF-IRES-puro between the AflII and XbaI sites. Starvation studies were carried out by transfecting cells containing vector or the FLIP gene with GFP-LC3, then 12-16 h post-transfection, treating the cells with Hank's solution for 4 hr or with 2 μM rapamycin for 3 hr. Autophagy was assessed by GFP-LC3 redistribution using an inverted fluorescence microscope, quantifying autophagy as means (±SD) of the combined results from three independent experiments. Autophagosomes were visualized through scanning electron microscopy. For electron microscopy imaging, cell pellets were fixed using a 0.1 M phosphate buffer (pH 7.4) containing 2% paraformaldehyde and 0.1% gluteraldehyde at room temperature for 1 hr. The cell pellets were then postfixed on ice for 2 hr for 1% osmium tetroxide and rinsed three times with distilled water. The fixed cell pellets were dehydrated by an ethanol (ETOH) dilution series of up to 100% ETOH and then immersed in propylene oxide (PO) for 2 min, with the cycle performed three times. The pellets were then infiltrated in a 3:1 PO/eponate resin mixture overnight and subsequently embedded in 100% eponate resin (Ted Pell Inc.) in beam capsules and allowed to harden overnight in a 65° C. oven. After hardening, tissue blocks were sectioned to a thickness of 70 nm and placed on 300 mesh copper grids. The grids were then counterstained with saturated uranyl acetate and lead citrate, then viewed through a Zeiss EM 10 electron microscope.

The ectopic expression of KSHV vFLIP, cFLIPₛ, HVS vFLIP, or MCV 159L in NIHI3T3 cells strongly suppressed starvation- and rapamycin-induced autophagy. Furthermore, electron microscopy to reveal the ultrastructures of autophagy as double-membraned vacuoles with visible cytoplasmic contents demonstrated that, while small differences in the basal number of autophagic vacuoles per cell were observed under normal conditions, under starvation conditions, a number of autophagic vacuoles were detected in NIH3T3-vector cells, but not in NIH3T3-KSHV-vFLIP cells.

During autophagy, an LC3 precursor (LC3-I) undergoes cleavage and lipidation to yield its processed form (LC3-II) (Mizushima et al. (2008) Nature 451:1069-1075). Immunoblotting was performed with an antibody against LC3 to further measure autophagic activity. NIH3T3-Vector, NIH3T3-KSHV-vFLIP, and NIH3T3-MCV-159L cells were treated with Hank's solution for 4 hr. Polypeptides were resolved by SDS-polyacrylamide gel electrophoresis (PAGE) and transferred to a PVDF membrane (Bio-Rad). Immunodetection was achieved with anti-tubulin (1:1000) (Santa Cruz Biotech) and anti-LC3. The proteins were visualized by a chemiluminescence reagent (Pierce) and detected by a Fuji Phosphor Imager. A large portion of LC3-I was processed to LC3-II in NIH3T3-vector cells under starvation conditions, while little or no processed LC3-II was detected in NIH3T3-KSHV-vFLIP, NIH3T3-MCV-159L, NIH3T3-HVS-vFLIP, and NIH3T3-cFLIPₛ cells. Finally, the ability of FLIP to block autophagy in multiple cell types was evaluated. At 12-16 hr post-transfection with GFP-LC3, HCT116, HaCat, and MEF cells containing vector or KSHV vFLIP were treated with 2 μM rapamycin for 3 hr. Subsequently, GFP-LC3 was detected using an inverted fluorescence microscope and the autophagy levels quantified as means (±SD) of the results from three independent experiments. KSHV vFLIP efficiently blocked autophagy in a number of cell types.

To investigate whether vFLIP expression suppresses autophagy in virus-infected cells, a KSHV-infected BCBL1 cell line was constructed (TREX-BCBL) in which a Flag-tagged KSHV-vFLIP gene was integrated into the chromosomal DNA under the control of a tetracycline-inducible promoter. (Nakamura et al. (2003) J. Virol. 77:4205-4220). At 12-16 hr post-transfection with GFP-LC3, TREX-BCBL-Vector and TREX-BCBL-vFLIP cells were treated with Doxycycline for 24 hr, followed by incubation with 2 μM rapamycin for an additional 12 hr. The detection of GFP-LC3 and autophagy levels was performed as described above, vFLIP expression was detected by IB with anti-Flag, and autophagosomes were visualized by scanning EM (scale bar=50 nm). Treatment of these cells with doxycycline strongly activated Flag-vFLIP expression. Following treatment with rapamycin, ectopic expression of vFLIP in TREX-BCBL cells extensively suppressed rapamycin-induced autophagy. EM studies also showed that, upon rapamycin treatment, the number of autophagic vacuoles significantly increased in TREX-BCBL-Vector cells but not in TREX-BCBL-vFLIP cells.

To determine the effects of the interactions of vFLIP with FADD, IKKαβγ, and 14-3-3σ on anti-autophagy activity, NIH3T3 cells stably expressing various vFLIP mutants were constructed: m14-3-3σ, which lacks 14-3-3σ-binding; mTRAF2, which lacks TRAF2-binding (Guasparri et al. (2006) EMBO Rep. 7:114-119); 67AAA and 58AAA, which have reduced NF-κB activation (Matta and Chaudhary (2004) Proc. Natl. Acad. Sci. USA 101:9399-9404); and mFADD, which lacks FADD-binding (Yang et al. (2005) Mol. Cell. 20:939-949). These cells were treated with either cyclohexamide (CHX) alone or CHX and TNF-α for 12 hr or with 2 μM rapamycin for 3 hr. Propidium iodide (PI) staining and flow cytometry analysis were performed to determine apoptosis levels. Alternatively, NIH3T3 cells containing vector, KSHV vFLIP, or its mutants were transfected with an NF-κB-luciferase reporter construct and a control renilla luciferase plasmid, pRL-SV40 (Promega). At 48 hr post-transfection, luciferase activity was measured with a luminometer using a dual luciferase assay kit (Promega) and normalized with renilla luciferase activity to determine transfection efficiency. GFP-LC3 puncta were detected using an inverted fluorescence microscope and the autophagy levels quantified as means (±SD) of the results from three independent experiments.

As previously shown, the 67AAA and 58AAA mutants showed considerably reduced NF-κB activation, while the 67AAA, 58AAA, and mFADD mutants either poorly blocked TNFα-induced apoptosis or no longer block it at all. (Matta and Chaudhary (2004) Proc. Natl. Acad. Sci. USA 101:9399-9404). In contrast, all of the vFLIP mutants were still capable of blocking starvation-induced autophagy as efficiently as vFLIP wild type, indicating that the anti-autophagy activity of KSHV vFLIP is genetically separable from its anti-apoptosis and NF-κB activation activities. KSHV vFLIP and cFLIPₛ both carried out NF-κB activation in addition to anti-apoptosis and anti-autophagy; MCV 159L did not carry out NF-κB activation but had anti-apoptotic and anti-autophagic activity; and HVS vFLIP had anti-autophagic activity.

### Example 2

### FLIP Interacts with Atg3

To identify cellular targets of KSHV-vFLIP specifically pertaining to its anti-autophagic activities, a yeast two-hybrid screen using a cDNA library from EBV-transformed human B-lymphocytes was performed. Yeast transformations with a cDNA library were performed using a method previously described. (Liang et al. (2006) Nat. Cell Biol. 8:688-699). Yeast strain Y187 bearing the Gal4-vFLIP fusion gene plasmid was grown overnight in synthetic dropout (SD)/-Trp medium to a density of approximately 10⁷ cells/ml, then diluted in 1 liter of warmed YPD to an optical density (OD600) of 0.2-0.3, and grown to an exponential stage. The cells were harvested and washed twice with 100 ml of water and once with TE (Clontech). The pellet was resuspended in 8 ml of 10 mM Tris-HCl (pH 7.5), 1 mM EDTA, and 0.1 M Li-acetate (LiOAc). The suspension was then mixed with 1 mg of transforming DNA and 20 mg of single-stranded salmon sperm DNA, after which 60 ml of a solution consisting of 40% polyethlyeneglycol-4000 in Tris-EDTA-LiOAc was added and thoroughly mixed, followed by incubation with agitation at 30° C. for 30 min. After a heat pulse at 42° C. for 15 min, the cells were pelleted, washed with 50 ml of Tris-EDTA, and plated on selective medium. Library screening and recovery of plasmids were performed according to the manufacturer's instructions (Clontech).

DNA sequence analysis revealed the Atg3 E2 enzyme of the LC3 conjugation system as a binding partner. The ability of FLIP to bind to Atg3 was evaluated by co-immunoprecipitation assay. HEK293T cells were transfected with Flag-vFLIP and/or GFP-Atg3 or Flag-vFLIP and/or GST-Atg3, and at 48 hr post-transfection, cells were harvested and lysed in an NP40 buffer supplemented with complete protease inhibitor cocktail (Roche). After pre-clearing with protein A/G agarose beads for 1 hr at 4° C., whole-cell lysates were used for immunoprecipitation with αFlag, αGFP, or GST. Generally, 1-4 μg of the antibody was added to 1 ml of the cell lysate and incubated at 4° C. for 8 to 12 hr. The immunoprecipitates were then washed extensively with a lysis buffer and eluted by boiling them with an SDS loading buffer for 5 min. Following immunoprecipitation, samples were treated to immunoblot using αAtg3 (1:500) (Novus). Co-immunoprecipitation showed that KSHV-vFLIP strongly interacted with endogenously and exogenously expressed Atg3, even in the presence of a 1M NaCl salt concentration. Additionally, cFLIPₛ, MCV 159L, and HVS vFLIP interacted with Atg3 as efficiently as KSHV-vFLIP.

The overall structure of yeast Atg3 has a unique hammer-like shape with the N-terminal and C-terminal regions discretely responsible for Atg7 and Atg8 (equivalent to mammalian LC3) binding, respectively. (Yamada et al. (2007) J. Biol. Chem. 282:8036-8043). Based on the structure of yeast Atg3, human Atg3 deletion mutants fused with a GST mammalian expression vector were constructed: GST-Atg3₁₋₉₃, GST-Atg3₁₉₃₋₂₆₈, GST-Atg3₂₆₈₋₃₁₅, and GST-Atg3₁₉₃₋₃₁₅-GST-tagged hATG3 and hATG3 mutant genes were cloned into a pcDNA5/FRT/TO derivative encoding an N-terminal GST epitope tag between the BamHI and NotI sites. GST pulldown with Flag-KSHV vFLIP, Flag-cFLIPₛ, Flag-MCV 159L, or Flag-HVS vFLIP along with GST-Atg3, GST-Atg3 N-terminal region (Nt), or GST-Atg3 C-terminal region (Ct) was performed as described above and was followed by immunoblotting with aFlag or aGST. GST pulldowns showed that vFLIP independently bound either the aa193-268 or aa268-315 region of the C-terminal of Atg3 in a manner almost identical to the binding of LC3 and Atg3. Furthermore, MCV 159L and HVS vFLIP demonstrated Atg3 binding patterns similar to KSHV vFLIP, while cFLIPₛ bound full-length Atg3 only.

Due to the substantial similarities between DEDI and DED2, Atg3 bound each DED of KSHV vFLIP with a similar affinity. Structural analyses have shown that the DED1 and DED2 of KSHV vFLIP and MCV 159L contain six and five α-helixes, respectively. (Yang et al. (2005) Mol. Cell. 20:939-949); Bagneris et al. (2008) Mol. Cell. 30:620-631). To further define the Atg3 binding sequences, each DED of KSHV vFLIP was divided into small α-helix fragments based on the previous structural analysis and expressed as mammalian GST fusions. Truncated mutant constructs of KSHV-vFLIP and hATG3 were created by subcloning the PCR products of cDNA fragments containing each domain of the associated genes into pcDNA5/FRT/TO. HEK293T cells were transfected with mutant constructs for GST pulldown as described above, followed by immunoblotting with αV5. The DED1α2 helix (10 aa) and the DED2 α4 helix (12 aa) of vFLIP were individually sufficient for Atg3 binding. However, while the deletion of either the DED1 α2 helix or the DED2 α4 helix of vFLIP showed no effect on Atg3 binding, deletion of both helix sequences (vFLIP mAtg3) considerably abrogated Atg3 binding. Additionally, a vFLIP mAtg3 mutant that no longer bound Atg3 was unable to block rapamycin-induced autophagy. In contrast, vFLIP mutants carrying a loss of 14-3-3σ, IKKαβγ complex, or FADD interaction bound Atg3 and blocked rapamycin-induced autophagy as efficiently as wild-type vFLIP. These results demonstrate that vFLIP and Atg3 efficiently interact with each other using two independent binding regions: Atg3 utilizes the aa193-268 and aa268-315 regions, while vFLIP uses the DED1 α2 helix and the DED2 α4 helix.

Due to identical Atg3 binding patterns seen in vFLIP and LC3, the ability of vFLIP to directly compete with LC3 for Atg3 binding was tested. HEK293T cells were transfected with GST-Atg3 and GFP-LC3 along with increasing amount of Flag-vFLIP. At 48 hr post-transfection, GST pulldown was performed as described above, followed by immunoblotting with αGFP or αFlag. Increasing vFLIP expression showed increased Atg3-vFLIP interaction, which led to the marked decrease of Atg3-LC3 interaction. Furthermore, vFLIP expression in NIH3T3 cells resulted in the dramatic reduction of endogenous Atg3 and LC3 interaction under normal conditions, although this interaction was weakly recovered upon treatment with 2 nM rapamycin for 12 hr.

### Example 3

### vFLIP Blocks Rapamycin-Induced Growth Suppression and Autophagic Death

The effect of vFLIP on autophagy was tested in a KSHV-infected BCBL1 cell line. TREX-BCBL-Vector and TREX-BCBL-vFLIP cells were treated with rapamycin or left untreated for 6 days in the presence of doxycycline and subjected to scanning EM, to examine the morphologies of over 100 dead cells and quantify cell death for apoptosis and autophagic death, or subjected to PI staining and cell cycle analysis. Rapamycin effectively induced growth suppression and cell death in KSHV-infected BCBL1 cells where its primary action appeared to be geared toward autophagic death. Cell cycle analysis showed minor increases of G1 cell cycle arrest and apoptosis (sub G1 population). In contrast, precise inspection via EM showed that approximately 60% of the dead cells induced by rapamycin carried intact nuclei, mitochondria, endoplasmic reticulum, and numerous large cytoplasmic membrane-bound vacuoles, which are characteristic of autophagic death.

To further demonstrate death by autophagy, siRNA was used to knockdown endogenous Beclin1 expression in BCBL1 cells. All siRNAs were produced by Dharmacon Research. siRNAs specific for human Beclin1 (5′AAGAUCCUGGACCGUGUCACC3′) and a nonspecific scrambled control siRNA were transfected using DharmaFect reagent (Dharmacon) according to the manufacturer's instructions. At 48-72 hr post-transfection, cells were analyzed for autophagy. Trypan blue staining and a Beckman Coulter Z2 Particle Count and Size analyzer were used to determine cell death (as a percentage) at day 5. Beclin1 siRNA significantly reduced endogenous Beclin1 expression in BCBLI cells, which subsequently attenuated rapamycin-induced autophagic death, whereas a control scrambled siRNA did not. Autophagic death was also induced by treatment with benzyloxycarbonylvalyl-alanyl-aspartic acid (O-methyl)-fluoro-methylketone (zVAD), a caspase inhibitor with a broad specificity and an autophagic death-inducing agent. (Yu et al. (2004) Science 304:1500-1502). zVAD effectively induced growth suppression and death of BCBL1 cells. However, ectopic expression of vFLIP wt, 58AAA or mFADD mutant in BCBL1 cells almost completely blocked rapamycin- or zVAD-induced growth suppression and autophagic death. However, the vFLIP mAtg3 mutant provided no protection for the BCBL1 cells from rapamycin-induced autophagy and autophagic death under identical conditions. Furthermore, HEK293 cells carrying the mutant KSHVΔvFLIP (Ye et al. (2008) J. Virol. 82:4235-4249) showed detectably reduced growth rates upon rapamycin treatment when compared to HEK293 cells carrying the wt KSHV.

### Example 4

### FLIP α2 and α4 Peptides Induce Autophagic Cell Death

The vFLIP α2 (10 aa) and α4 (12 aa) peptides that independently bound Atg3 at high efficiency were fused with the HIV-1 TAT protein transduction domain for intracellular delivery (Gump and Dowdy (2007) Trends Mol. Med. 13:443-448) and tested for their potential effects on autophagy induction. The K-α2 and K-α4 peptides contained KSHV vFLIP aa20-29 and aa128-139 regions, respectively, as retro-inverso versions to circumvent the proteolytic degradation. Mutant peptides K-α2m and K-α4m were also included, which replaced the hydrophobic core residues of the K-α2 (L₂₁F₂₂L₂₃) and K-α4 (F₁₃₀L-WVY₁₃₉) peptides with alanines, as well as the C-α2 and C-α4 peptides containing the cFLIPₛ regions aa19-28 and aa128-139, respectively. At 12 hr post-transfection with GFP-LC3, KSHV-infected TREX-BCBL-Vector and TREX-BCBL-vFLIP cells were incubated with various concentrations of the peptides or rapamycin for an additional 12 hr, followed by an assessment of the GFP-LC3 puncta and cell death. Either 30 or 50 μM of the K-α2, K-α4, C-α2, or C-α4 peptide was able to induce autophagy in TREX-BCBL-Vector cells as effectively as 2 μM rapamycin. Furthermore, overnight incubation of TREX-BCBL-Vector cells with 30 or 50 μM of the K-α2, K-α4, C-α2, or C-α4 peptide robustly induced cell death: 50-60% cell death occurred with the K-α2 and C-α2 peptides while 75-90% cell death occurred with the K-α4 and C-α4 peptides. In contrast, treatment with HIV-1 TAT only, the K-α2m or the K-α4m mutant peptide showed no increase in autophagy nor cell death under identical conditions. EM studies showed that a vast majority of KSHV-infected BCBL1 cells underwent autophagic death, with remarkably large numbers of sizeable autophagic vacuoles in their cytoplasmic regions, while a small population of cells exhibited both autophagic and apoptotic deaths. Peptide-treated TREX-BCBL-Vector and TREX-BCBL-vFLIP cells were subjected to immunoblotting with αLC3 and aactin. A high amount of LC3-II was detected in K-α2 and K-α4 treated BCBL1 cells.

Levels of phosphorylation of p70S6K and ribosomal protein S6 in BCBL1 cells were evaluated by immunoblotting with α-p70S6K, aphospho-specific p70S6K, aphospho-specific S6, or αactin. The inhibition of mTor kinase activity was evidenced by the near complete loss of the phosphorylation of p70S6K and ribosomal protein S6 in rapamycin-treated BCBL1 cells. In contrast, the phosphorylation of p70S6K and ribosomal protein S6 exhibited marginal reduction or remained unaffected, respectively, in K-α2 or K-α4 peptide-treated BCBL1 cells. While KSHV vFLIP expression readily blocked rapamycin-induced autophagy and cell death, its inhibition of peptide-induced autophagy and cell death was minimal. Finally, incubation with the α2 and α4 peptides pronouncedly blocked the interaction between Atg3 and cFLIPₛ or vFLIP in a dosage dependent manner, suggesting that the FLIP α2 and α4 peptides efficiently compete with the full-length FLIP protein for Atg3 binding, perhaps antagonizing FLIP activity to lead to robust autophagic death.

The potential cell-type specificity of the activity of the vFLIP peptides was assessed by treating virus-associated or non-virus-associated lymphoma cells with 30 or 50 μM of the K-α2 or the K-α4 peptide overnight and subsequently assessing death levels. The K-α2 peptide primarily targeted KSHV-infected PEL cells (BCBL1, BCP-1, and BC-3), but not KSHV/EBV-coinfected PEL cells (BC-1 and JSC-1), EBV-immortalized Bob-B cells, nor non-virus-associated lymphoma cells (BJAB, K562 and Jurkat-T). In striking contrast, the K-α4 peptide broadly targeted most lymphoma cells, resulting in varying degrees of cell death. Finally, a combinatory treatment of KSHV-infected BCBL1 cells with 25 nM rapamycin and 20 μM of the K-α2 or the K-α4 peptide led to a marked increase of growth suppression and cell death compared to treatment with rapamycin or the individual peptides alone.

### Example 5

### Pre-Clincal Testing of the FLIP Peptides and Small Molecules with a Mouse Xenografting Platform

To demonstrate that engraftment and growth of virus-induced BCBL1 lymphoma cells or acute lymphoblastic leukemia cells can be monitored in vivo at high sensitivity, these cells are transduced with a lentiviral construct encoding firefly luciferase before injection. Luciferase activity is measured in two xenografted mice 8, 12 and 15 days after injection. Whereas engraftment as judged by FACS analysis of peripheral blood is not seen until after three weeks post-injection, all mice will show engraftment after 12 days via the bioluminescence analysis. The bioluminescent signal can be quantified distinguishing signal originating from whole body, hips and femur and spleen. Using this mouse xenografting platform, the efficacy of the FLIP peptides and their derivative small molecules are assayed for the suppression of ALL cells in vivo.

The lentivirus-luciferase infected 2.5×10⁶ primary human ALL cells are injected into NOD/SCIDγc^{-/-} recipient mice by tail-vein injection. Or the lentivirus-luciferase infected 2.5×10⁶ virus-induced BCBL1 lymphoma cells are injected into NOD/SCIDyc^{-/-} recipient mice by intraperitoneal injection. After a week, mice are be implanted with osmotic minipumps for continuous delivery of 100 mg/kg/d FLIP peptide. The FLIP mutant peptides are included as negative controls. Mice will be monitored by bioluminescence analysis. To further test the effects of the FLIP peptide, ALL or BCBL1 cells are mixed with peptide or small molecule right before the injection. DMSO will be used as a control. Bioluminescence analysis is used to monitor the xenografting activity in NOD/SCIDyc^{-/-} recipient mice. Diseased mice are humanely killed, at which point spleen, bone marrow and peripheral blood are harvested for flow cytometric analysis. CBC analysis is performed. If prolonged survival in the FLIP peptide treated group is observed, it will determined whether this beneficial effect is due to the autophagy induction of drug resistant ALL or BCBL1 cells. At least 6 mice per experimental group are transplanted and injected with the FLIP peptide; results from 4 to 5 independent transplantations will be analyzed separately and pooled.

### Example 6

### Alternating Lever Cyclic Ratio Rat Model for the Treatment of Alzheimer's Disease

A preparation of a FLICE-like inhibitor protein, peptide fragment or composition described herein may be assayed as a potential therapeutic compound for the treatment of Alzheimer's disease (AD) using the Alternating Lever Cyclic Ratio (ALCR) rat model of AD. This assay is used to show in vivo efficacy. This highly sensitive model has been able to detect cognitive deficits due to direct injection of cell-derived Aβ oligomers into rat brain. Using this technique, a direct injection of amyloid β-derived diffusible ligands (ADDLs) made from synthetic Aβ₁₋₄₂ peptides and the therapeutic FLICE-like inhibitor protein, peptide fragment or composition using the ALCR procedure may be tested.

The ALCR test has proven to be much more sensitive than previously published methods for measuring drug effects on cognitive function. In this task, rats must learn a complex sequence of lever-pressing requirements in order to earn food reinforcement in a two-lever experimental chamber. Subjects must alternate between two levers by switching to the other lever after pressing the first lever enough to get food reward. The exact number of presses required for each food reward changes, first increasing from 2 responses per food pellet up to 56 presses per food pellet, then decreasing back to 2 responses per pellet. Intermediate values are based on the quadratic function, x²−x. One cycle is an entire ascending and descending sequence of these lever press requirements (e.g., 2, 6, 12, 20, 30, 42, 56, 56, 42, 30, 20, 12, 6, and 2 presses per food reward). Six such full cycles are presented during each daily session. Errors are scored when the subject perseveres on a lever after pressing enough to get the food reward, i.e., does not alternate (a Perseveration Error), or when a subject switches levers before completing the response requirement on that lever (an Approach Error).

### Materials and Methods

Synthetic Aβ₁₋₄₂ powder is dissolved in 1,1,1,3,3,3 hexafluorisopropanol (HFIP) to afford a solution of Aβ₁₋₄₂ in HFIP of about 1 mM and allowed to incubate at ambient temperature for about 1 h. The resulting solution is chilled on ice for about 5-10 min, then aliquoted into eppendorf tubes to provide about 50 μL of solution per tube. The tubes are then placed in a chemical fume hood and allowed to stand overnight to allow the HFIP to evaporate under a slow stream of nitrogen. To remove final traces of HFIP, the tubes are subjected to two SpeedVac cycles of 15 min at room temperature and about 15 to 25 mm Hg of vacuum. The resulting films of monomerized Aβ₁₋₄₂ peptide are stored over desiccant at −80° C. until used.

A tube of monomerized Aβ₁₋₄₂ peptide is warmed to room temperature and the Aβ₁₋₄₂ peptide is dissolved in anhydrous DMSO to afford a peptide stock DMSO solution containing about 10 μM to about 100 μM Aβ₁₋₄₂ peptide in DMSO.

A predetermined amount of test FLICE-like inhibitor protein, peptide fragment or composition stock solution in anhydrous DMSO is added to about 998 μL of neural basal media (phenol red free, Gibco 12348-017) to give a compound neural basal media solution containing about 10 to about 100 μM of test protein, peptide fragment or composition in neural basal media.

For the Aβ₁₋₄₂ peptide only treatment, peptide stock DMSO solution is added to 37° C. neural basal to obtain the requisite Aβ₁₋₄₂ peptide monomer concentration, provided that the maximum concentration of DMSO is 1% or less, and the tube is vortexed for 30 to 60 seconds, spun down briefly in a microfuge and incubated at 37° C. for 15 min prior to the start of injections.

For the Aβ₁₋₄₂ peptide plus test FLICE-like inhibitor protein, peptide fragment or composition treatment, peptide stock DMSO solution is added to 37° C. compound neural basal media solution to obtain the requisite Aβ₁₋₄₂ peptide monomer concentration, provided that the maximum concentration of DMSO is 1% or less, and the tube is vortexed for 30 to 36 seconds, spun down briefly in a microfuge and incubated at 37° C. for 15 min prior to the start of injections.

For control injections, compound neural basal media solution is incubated at 37° C. for 15 min prior to the start of injections.

Rats: Rats are trained under ALCR until their error rates are stable. Once the rats are placed upon the final ALCR procedure, training sessions are conducted 7 days each week until the end of the study.

Surgery: All rats receive a single 28 ga cannula, which is permanently affixed to the skull, and aimed at the lateral ventricle. Half the rats receive cannula in the right ventricle and half receive cannula in the left ventricle. Rats are allowed 5 days to recover from surgery before training resumed.

Injection of Test Material and ALCR Testing: Test are conducted about every fourth day. Animals received a 20 μL injection of control, peptide, or peptide plus compound solutions via the implanted cannula over about 3 to 4 minutes. Animals are tested about 3 hours following injection.

Error Rate Analysis: All error rates under will be compared to baseline error rates consisting of at least 3 non-treatment days temporally contiguous to the injection. Student's T test of statistical inference was used for analysis of effects.

### Contemplated Results

It is contemplated that a significant increase in Perseveration Error rate will be found between baseline error rates and error rates produced by Aβ₁₋₄₂ ADDLs. It is also contemplated that addition of one or more of the FLICE-like inhibitor protein, peptide fragment or composition described herein will not increase error rates when given alone, but when combined with the Aβ₁₋₄₂ ADDLs, the compound(s) will eliminate the increase in Perseveration Errors. Thus, it is contemplated that at least one or more of the FLICE-like inhibitor protein, peptide fragment or composition described herein will rescued errors produced by Aβ₁₋₄₂ ADDLs.

### Example 7

### FLIP Peptides Induced Cell Death of Cancer Cells

This example tested the anticancer activities of the peptides of the invention in various cancer cells, including ALL cells, a prostate cancer cells and colon cancer cells.

Tumor cells were treated with peptides at various concentrations for 12 hours and their viability measured by trypan blue exclusion, followed by analyses using the Beckman Coulter Z2 Particle Count and Size Analyzer (BC Z2 CS Analyzer). A minimum of 100 cells per sample using triplicate samples was counted per condition per experiment.

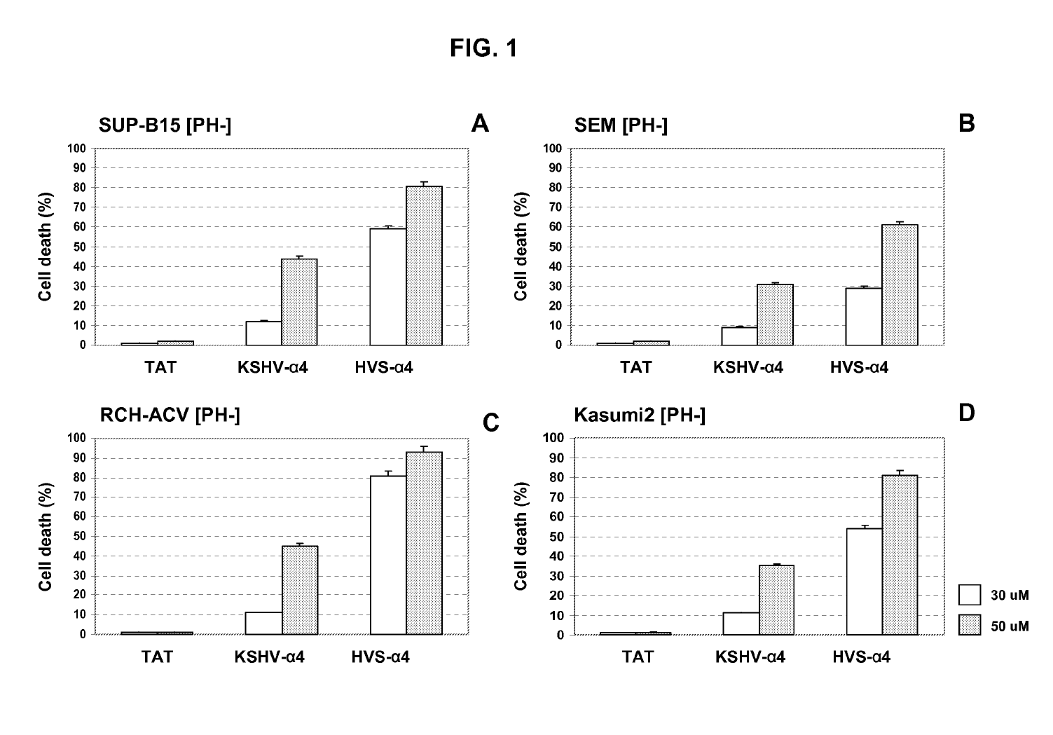

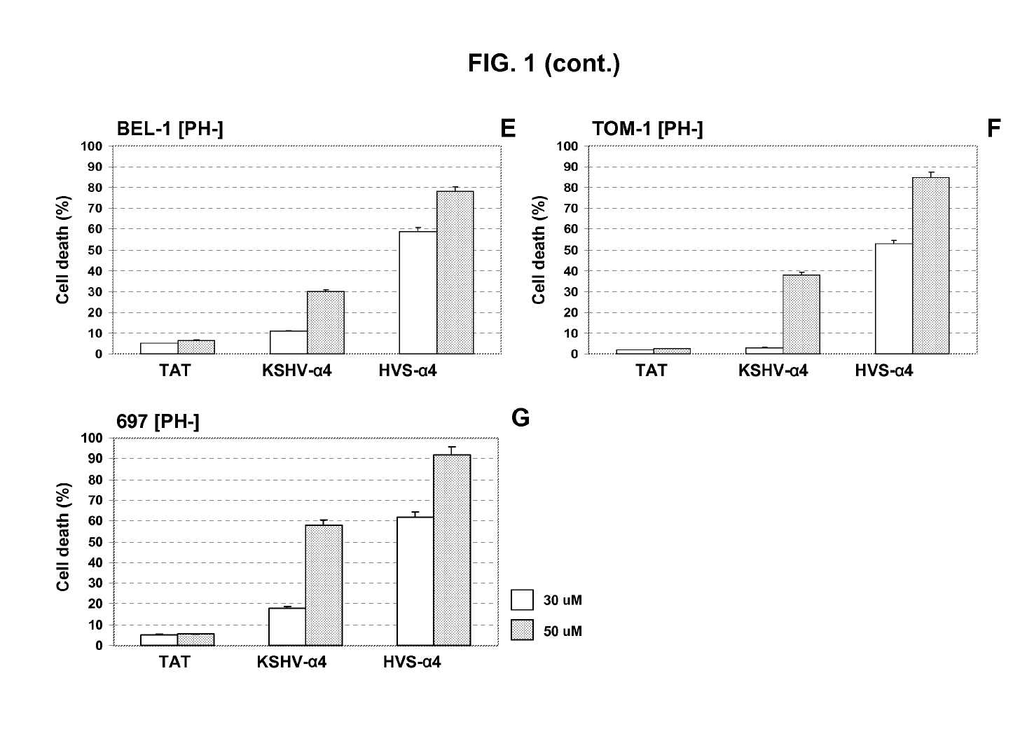

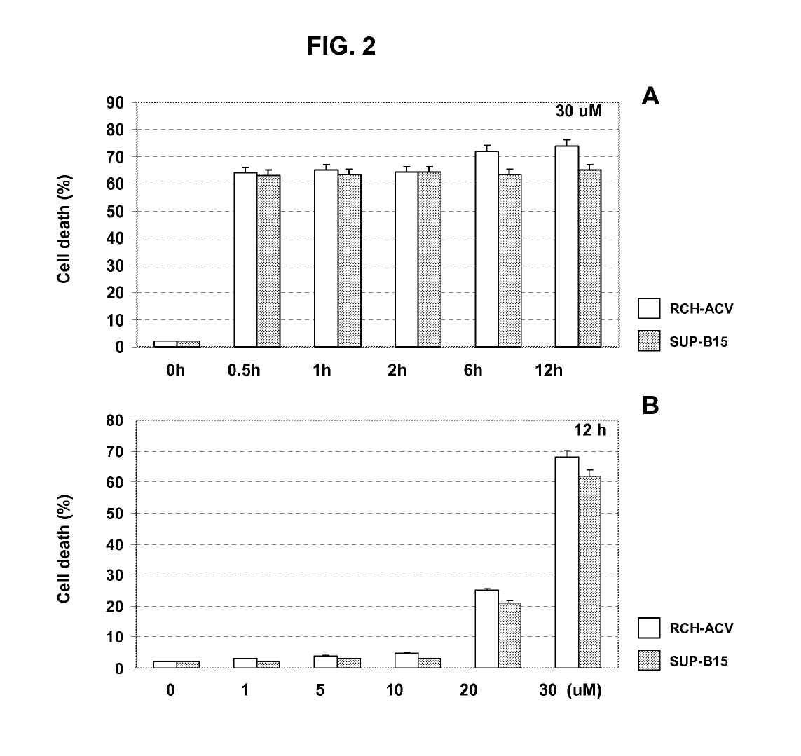

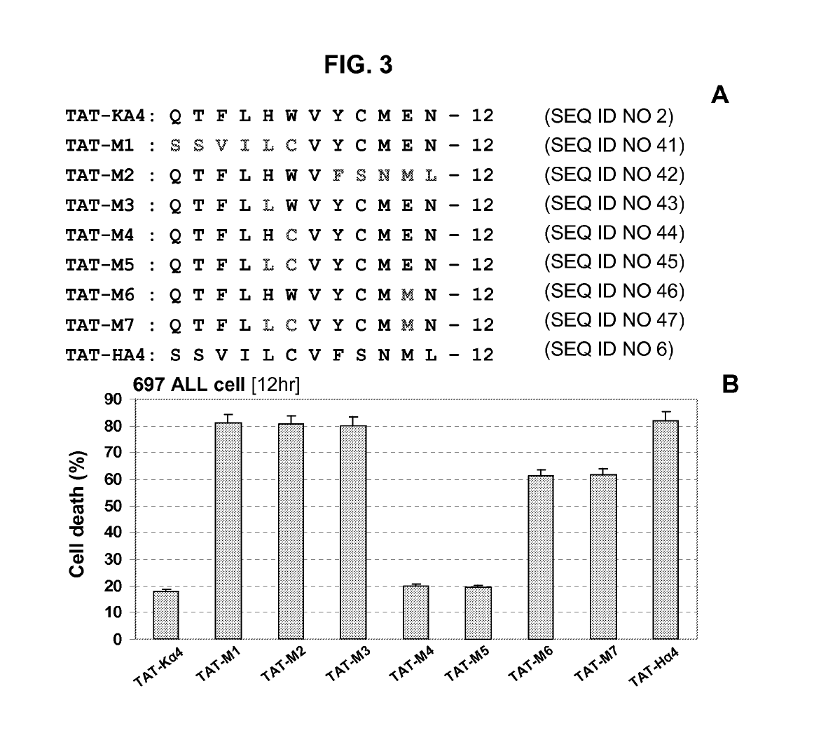

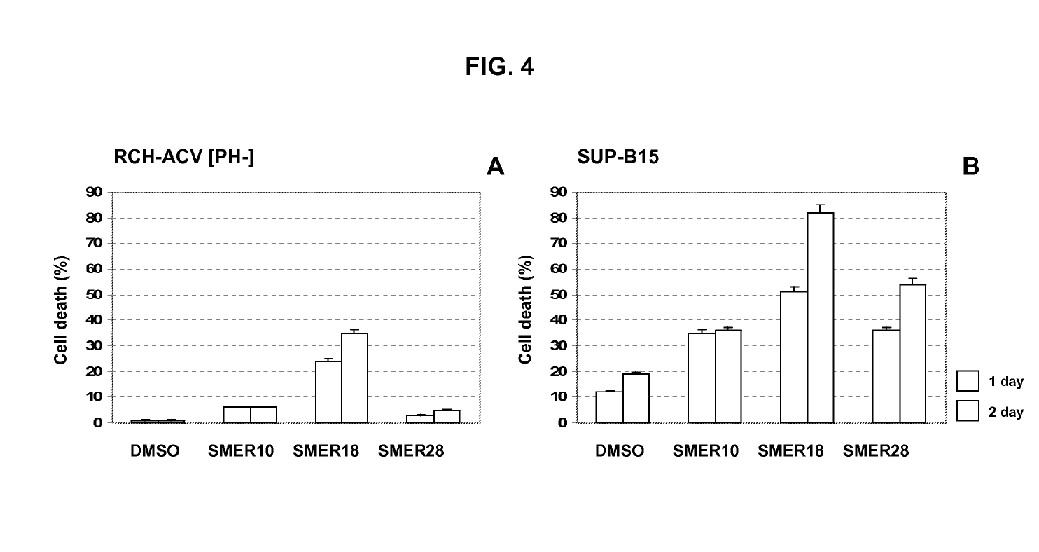

As shown in FIG. 1A-G, compared to TAT alone, TAT linked KSHV-α4 and HVS-α4 induced cell deaths in various ALL cells. FIG. 2 demonstrates that HVS-α4 induced cell death occurred at least as early as half an hour (A) and was dose dependent (B). FIG. 3 shows the sequences of a number of hybrids and variants of KSHV-α4 (Kα4) and HVS-α4 (H-α4) and their anticancer activities. Similar anti-cancer activities have also been observed for SMER (small molecule enhancer of rapamycin) as disclosed in Sarkar et al. (2007) Nature Chemical Biology 3:331. FIG. 4 shows the anti-cancer activity of SMER (small molecule enhancer of rapamycin) as disclosed in Sarkar et al. (2007) Nature Chemical Biology 3:331, in ALL cells, in RCH-AcV and in SUP-B15.

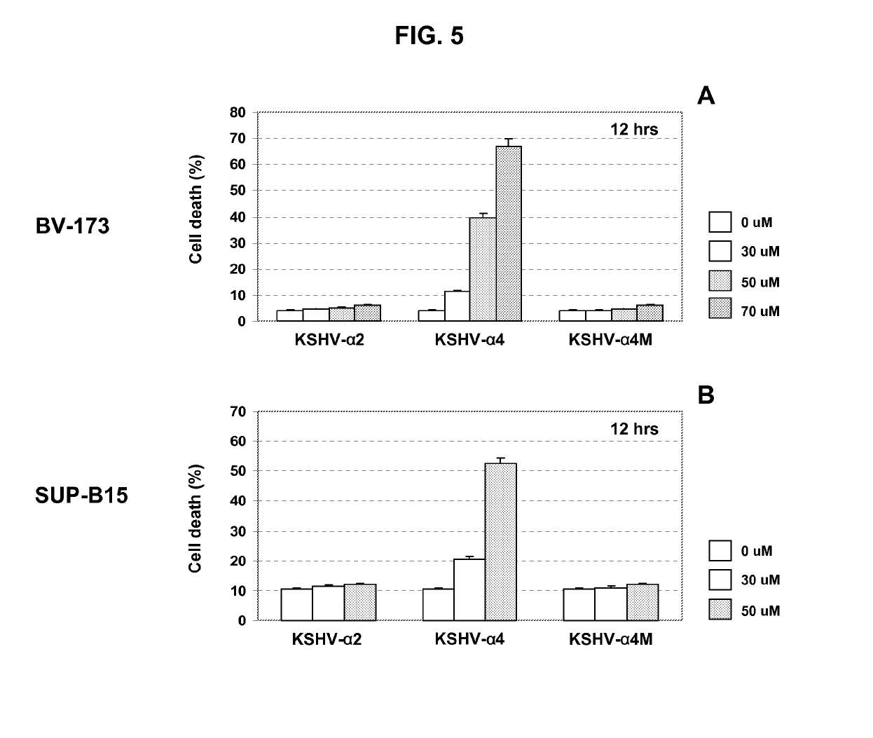

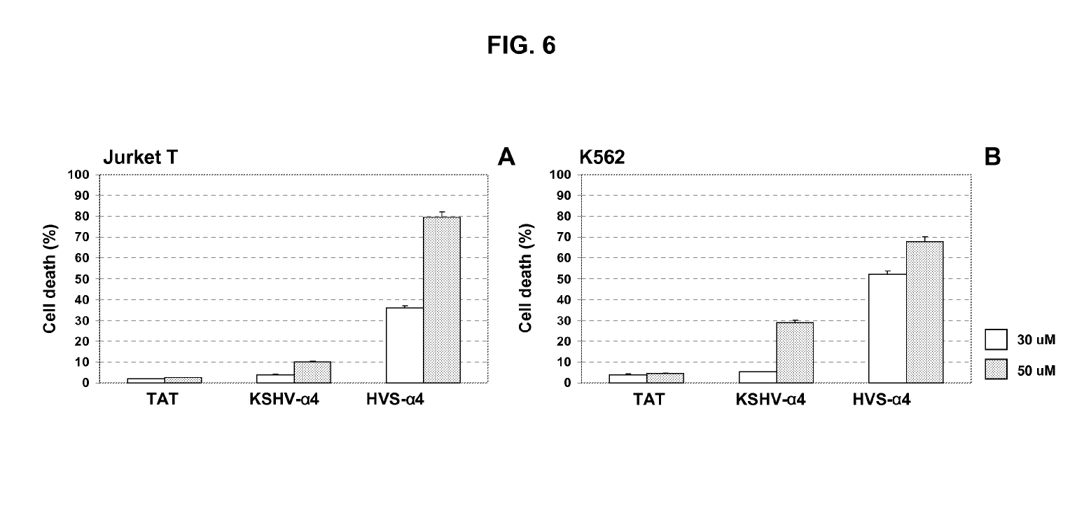

FIG. 5 shows that KSHV-α2, α4 and α4M all had significant anticancer activities in ALL cell lines. FIG. 6 further shows KSHV-α4 and HVS-α4 induced cell death in human T cell lymphoblast-like cells, Jurket T, and human erythromyeloblastoid leukemia cells, K562.

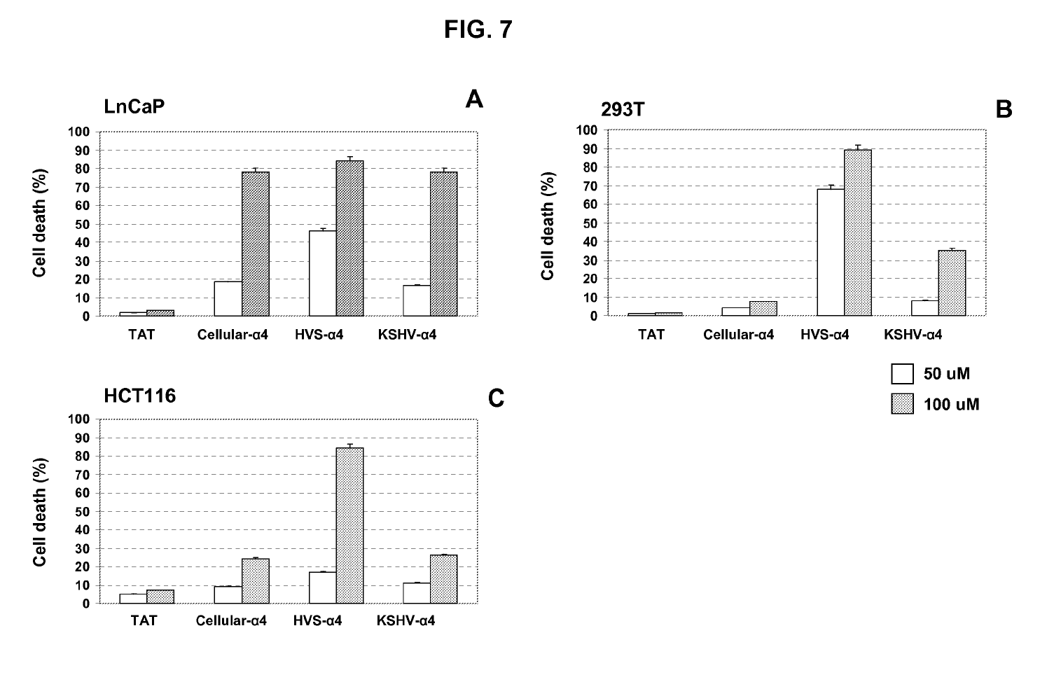

Anticancer activities of the FLIP peptides were further observed in solid tumor cells. In FIG. 7, anticancer activities of cellular-α4, HVS-α4, KSHV-α4 were observed with LNCap cells, a prostate cancer line, 293T cells and HCT116, a colon cancer cell line.

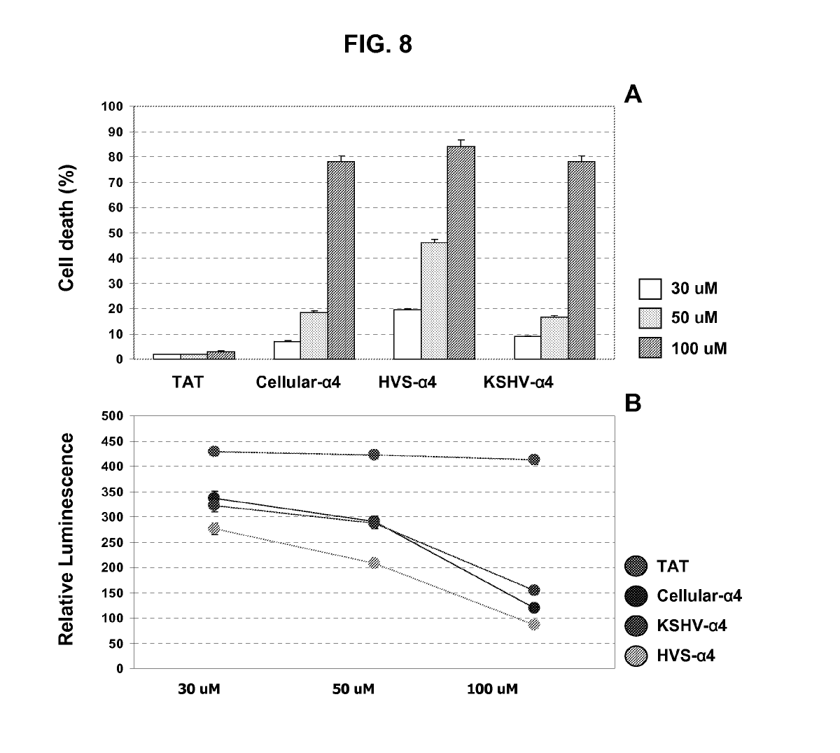

FIG. 8 further confirms the anticancer activities of cellular-α4, HVS-α4 and KSHV-α4 in LNCaP cells.

This example, therefore, demonstrates the anticancer activities of FLIP α2 and α4 peptides of the present invention in leukemia as well as in solid tumor cells.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

1. A method for increasing or inducing the death of a cancer cell comprising administering to the cell an effective amount of one or more of: a FLIP peptide fragment, variant, hybrid or biological equivalent thereof, an amino acid comprising SEQ ID. NOS. 1 through 8, 15 through 18, 41 through 47 or a biological equivalent of each thereof, a FLIP peptide fragment isolated from a FLIP protein death effector domain region of the FLIP protein or from a portion of the death effector domain region of the FLIP protein, a FLIP peptide fragment isolated from an alpha-helix fragment of the FLIP protein death effector domain region or from a portion of the alpha-helix fragment of the FLIP protein death effector domain region, or a peptide selected from one or more of:
(a) an isolated or recombinant peptide comprising one or more peptide shown in SEQ ID NO. 41 through 47, or an amino acid sequence having at least 80% homology to an amino acid sequence of SEQ ID NO. 41 to 47, wherein amino acid 5 and/or 6 is Lor W, respectively;
(b) an isolated or recombinant variant peptide comprising an amino acid sequence selected from SEQ ID NOS. 2, 4, 6 or 8, having a substitution with 1, 2, 3, or 4 amino acids at the corresponding positions of the a region of one or more other peptides selected from SEQ ID NOS. 2, 4, 6 or 8;
(c) an isolated or recombinant peptide comprising a hybrid peptide between any two amino acid sequences selected from SEQ ID NO.2, 4, 6 or 8 or a peptide having at least 80% homology to the hybrid peptide;
(d) an isolated or recombinant hybrid peptide comprising anN-terminal amino acid sequence of SEQ ID NO. 1 to 8 and a C-terminal fragment from a second non-identical amino acid sequence of SEQ ID NO. 1 to 8;
(e) an isolated or recombinant peptide comprising the amino acid sequence of any one of SEQ ID NO. 41 to 47, or a variant, hybrid or biological equivalent of each thereof; or
(f) an isolated or recombinant retro-inverso peptide of the peptide of any one of (a) to (e).

2. The method of claim 1, wherein the contacting is in vitro or in vivo.

3. The method of claim 1, wherein the FLIP peptide fragment comprises one or more amino acid sequence of the group of a peptide comprising an amino acid comprising SEQ ID. NOS. 1 through 8 or 15 through 18; a biologically equivalent peptide fragment of an amino acid comprising SEQ ID. NOS. 1 through 8 or 15 through 18; a peptide having at least 80% homology to an amino acid of SEQ ID NOS. 1 through 8 or 15 through 18; a FLIP peptide fragment isolated from a FLIP protein death effector domain region of the FLIP protein or from a portion of the death effector domain region of the FLIP protein; and a FLIP peptide fragment isolated from an alpha-helix fragment of the FLIP protein death effector domain region or from a portion of the alpha-helix fragment of the FLIP protein death effector domain region.

4. The method of claim 1, wherein the isolated FLIP peptide fragment comprising two non-contiguous death effector domain regions of cFLIP comprising the amino acid sequences EVVLFLLNVF (SEQ ID NO.1) and QTFLHWVYCMEN (SEQ ID NO.2), or amino acid sequences having at least 80% homology to SEQ ID NOS. 1 and 2 or a biologically equivalent to SEQ ID NOS. 1 and 2.

5. The method of claim 1, wherein the isolated FLIP peptide fragment comprises two non-contiguous death effector domain regions of cFLIP, comprising the amino acid sequences EMLLFLCRDV (SEQ ID NO.3) and KSFLDLVVELEK (SEQ ID NO.4), or amino acid sequences having at least 80% homology to SEQ ID NOS. 3 and 4 or a biologically equivalent to SEQ ID NOS. 3 and 4.

6. The method of claim 1, wherein the isolated FLIP peptide fragment comprises two non-contiguous death effector domain regions of cFLIP, comprising the amino acid sequences YCLLFLINGC (SEQ ID NO.5) and SSVILCVFSNML (SEQ ID NO.6), or amino acid sequences having at least 80% homology to SEQ ID NOS. 5 and 6 or a biologically equivalent to SEQ ID NOS. 5 and 6.

7. The method of claim 1, wherein the isolated FLIP peptide fragment comprises two non-contiguous death effector domain regions of cFLIP, comprising the amino acid sequences SLLLFLCHDA (SEQ ID NO. 7) and SRFVELVLALEN (SEQ ID NO. 8), or amino acid sequences having at least 80% homology to SEQ ID NOS. 7 and 8 or a biologically equivalent to SEQ ID NOS. 7 and 8.

8. The method of claim 1, wherein the cell is selected from a leukemia, a lymphoma a lung tumor, a non-small cell lung tumor, a breast tumor cell, a head and neck tumor, an ovarian tumor, a colon tumor, a rectal tumor, a colorectal tumor, an esophageal tumor, a gastric tumor, a liver tumor, a bone tumor, a spleen tumor, a pancreatic tumor, or a gallbladder tumor.

9. A method for treating cancer in a subject in need thereof, comprising administering to the subject an effective amount of one or more of: a FLIP peptide fragment, variant, hybrid or biological equivalent thereof, an amino acid comprising SEQ ID. NOS. 1 through 8, 15 through 18, 41 through 47 or a biological equivalent of each thereof, a FLIP peptide fragment isolated from a FLIP protein death effector domain region of the FLIP protein or from a portion of the death effector domain region of the FLIP protein, a FLIP peptide fragment isolated from an alpha-helix fragment of the FLIP protein death effector domain region or from a portion of the alpha-helix fragment of the FLIP protein death effector domain region, or a peptide selected from one or more of:
(a) an isolated or recombinant peptide comprising one or more peptide shown in SEQ ID NO. 41 through 47, or an amino acid sequence having at least 80% homology to an amino acid sequence of SEQ ID NO. 41 to 47, wherein amino acid 5 and/or 6 is Lor W, respectively;
(b) an isolated or recombinant variant peptide comprising an amino acid sequence selected from SEQ ID NOS. 2, 4, 6 or 8, having a substitution with 1, 2, 3, or 4 amino acids at the corresponding positions of the a region of one or more other peptides selected from SEQ ID NOS. 2, 4, 6 or 8;
(c) an isolated or recombinant peptide comprising a hybrid peptide between any two amino acid sequences selected from SEQ ID NO.2, 4, 6 or 8 or a peptide having at least 80% homology to the hybrid peptide;
(d) an isolated or recombinant hybrid peptide comprising anN-terminal amino acid sequence of SEQ ID NO. 1 to 8 and a C-terminal fragment from a second non-identical amino acid sequence of SEQ ID NO. 1 to 8;
(e) an isolated or recombinant peptide comprising the amino acid sequence of any one of SEQ ID NO. 41 to 47, or a variant, hybrid or biological equivalent of each thereof; or
(f) an isolated or recombinant retro-inverso peptide of the peptide of any one of (a) to (e), thereby treating cancer in the subject.

10. The method of claim 9, wherein the FLIP peptide fragment comprises one or more amino acid sequence of the group of a peptide comprising an amino acid comprising SEQ ID. NOS. 1 through 8 or 15 through 18; a biologically equivalent peptide fragment of an amino acid comprising SEQ ID. NOS. 1 through 8 or 15 through 18; a peptide having at least 80% homology to an amino acid of SEQ ID NOS. 1 through 8 or 15 through 18; a FLIP peptide fragment isolated from a FLIP protein death effector domain region of the FLIP protein or from a portion of the death effector domain region of the FLIP protein; and a FLIP peptide fragment isolated from an alpha-helix fragment of the FLIP protein death effector domain region or from a portion of the alpha-helix fragment of the FLIP protein death effector domain region.

11. The method of claim 9, wherein the isolated FLIP peptide fragment comprising two non-contiguous death effector domain regions of cFLIP comprising the amino acid sequences EVVLFLLNVF (SEQ ID NO. 1) and QTFLHWVYCMEN (SEQ ID NO.2), or amino acid sequences having at least 80% homology to SEQ ID NOS. 1 and 2 or a biologically equivalent to SEQ ID NOS. 1 and 2.

12. The method of claim 9, wherein the isolated FLIP peptide fragment comprises two non-contiguous death effector domain regions of cFLIP, comprising the amino acid sequences EMLLFLCRDV (SEQ ID NO.3) and KSFLDLVVELEK (SEQ ID NO.4), or amino acid sequences having at least 80% homology to SEQ ID NOS. 3 and 4 or a biologically equivalent to SEQ ID NOS. 3 and 4.

13. The method of claim 9, wherein the isolated FLIP peptide fragment comprises two non-contiguous death effector domain regions of cFLIP, comprising the amino acid sequences YCLLFLINGC (SEQ ID NO.5) and SSVILCVFSNML (SEQ ID NO.6), or amino acid sequences having at least 80% homology to SEQ ID NOS. 5 and 6 or a biologically equivalent to SEQ ID NOS. 5 and 6.

14. The method of claim 9, wherein the isolated FLIP peptide fragment comprises two non-contiguous death effector domain regions of cFLIP, comprising the amino acid sequences SLLLFLCHDA (SEQ ID NO. 7) and SRFVELVLALEN (SEQ ID NO. 8), or amino acid sequences having at least 80% homology to SEQ ID NOS. 7 and 8 or a biologically equivalent to SEQ ID NOS. 7 and 8

15. The method of claim 9, wherein the cancer is selected from a leukemia, a lymphoma a lung tumor, a non-small cell lung tumor, a breast tumor cell, a head and neck tumor, an ovarian tumor, a colon tumor, a rectal tumor, a colorectal tumor, an esophageal tumor, a gastric tumor, a liver tumor, a bone tumor, a spleen tumor, a pancreatic tumor, or a gallbladder tumor.

16. The method of claim 1, wherein the effective amount causes at least about 75% effectiveness when applied in a molar concentration of less than about 10 micromolar as compared to a control that does not receive the composition.

17. An isolated or recombinant peptide comprising one or more peptide shown in SEQ ID NO. 41 through 47, or an amino acid sequence having at least 80% homology to an amino acid sequence of SEQ ID NO. 41 to 47, wherein amino acid 5 and/or 6 is Lor W, respectively.

18. An isolated or recombinant variant peptide comprising an amino acid sequence selected from SEQ ID NOS. 2, 4, 6 or 8, having a substitution with 1, 2, 3, or 4 amino acids at the corresponding positions of the a region of one or more other peptides selected from SEQ ID NOS. 2, 4, 6 or 8.

19. An isolated or recombinant peptide comprising a hybrid peptide between any two amino acid sequences selected from SEQ ID NO.2, 4, 6 or 8 or a peptide having at least 80% homology to the hybrid peptide.

20. An isolated or recombinant hybrid peptide comprising an N-terminal amino acid sequence of SEQ ID NO. 1 to 8 and a C-terminal fragment from a second non-identical amino acid sequence of SEQ ID NO. 1 to 8.

21. An isolated or recombinant peptide comprising the amino acid sequence of any one of SEQ ID NO. 41 to 47, or a variant, hybrid or biological equivalent of each thereof.

22. An isolated or recombinant retro-inverso peptide of the peptide of claim 17.

23-24. (canceled)

25. An antibody that specifically recognizes and binds the peptide of claim 17.

26-29. (canceled)

30. A method of increasing or inducing autophagy in a cell, comprising contacting with cell with an effective amount of a peptide of claim 17.

31-34. (canceled)

35. A screen to identify possible therapeutic agents comprising contacting a first cell or tissue sample with a test agent and a second cell or tissue agent with one or more of a FLIP peptide fragment, variant, hybrid or biological equivalent thereof, an amino acid comprising SEQ ID. NOS. 1 through 8, 15 through 18, 41 through 47 or a biological equivalent of each thereof, a FLIP peptide fragment isolated from a FLIP protein death effector domain region of the FLIP protein or from a portion of the death effector domain region of the FLIP protein; and a FLIP peptide fragment isolated from an alpha-helix fragment of the FLIP protein death effector domain region or from a portion of the alpha-helix fragment of the FLIP protein death effector domain region, each under conditions that promote autophagy of the cell or in the tissue, and comparing the autophagy of the first sample with the second sample, wherein the test agent is a possible therapeutic agent if the autophagy of the first sample is the same or similar to the second sample, thereby screening for possible therapeutic agents.
